(19) United States
(12) Patent Application Publication
(10) Pub. No.: US 2026/0315803 A1
(43) Pub. Date: Oct. 15, 2026

(54) SELECTIVE NERVE FIBER STIMULATION FOR THERAPY

(71) Applicant: Medtronic, Inc., Minneapolis, MN (US)

(72) Inventors: Leonid M. Litvak, Bet Shemesh (IL); Jeffrey Edward Arle, Concord, MA (US); David A. Dinsmoor, North Oaks, MN (US); Kristen W. Carlson, Concord, MA (US)

(21) Appl. No.: 18/998,830

(22) PCT Filed: Jul. 27, 2023
(86) PCT No.: PCT/US2023/071141

§ 371 Date: Jan. 27, 2025

Related U.S. Application Data
(60) Provisional application No. 63/392,804, filed on Jul. 27, 2022.

(51) Int. Cl. A61N 1/36

(52) U.S. Cl. CPC A61N 1/36139; A61N 1/36062; A61N 1/36157; A61N 1/36164; A61N 1/36171; A61N 1/36178; A61N 1/36185

(57) ABSTRACT

Systems, devices, and techniques are described for determining stimulation parameter values based on a latency determined from an evoked compound action potential (ECAP). In one example, processing circuitry is configured to control delivery of a first burst of pulses, each pulse of the first burst of pulses defined by a first parameter set, control delivery of a stimulation pulse defined by a second parameter set; receive information representative of an ECAP signal elicited by the stimulation pulse, determine an ECAP characteristic of the ECAP signal, the ECAP characteristic indicative of a latency of one of more nerve fibers activated by the stimulation pulse, determine, based on the ECAP characteristic, one or more parameter values of the first parameter set to generate a third parameter set for a second burst of pulses, and control delivery of the second burst of pulses according to the third parameter set.

This application is a PCT application that claims priority to and the benefit of U.S. Provisional Patent Application No. 63/392,804, filed Jul. 27, 2022, the entire contents of which is incorporated herein by reference.

### TECHNICAL FIELD

This disclosure generally relates to electrical stimulation, and more specifically, control of electrical stimulation.

### BACKGROUND

Medical devices may be external or implanted and may be used to deliver electrical stimulation therapy to patients via various tissue sites to treat a variety of symptoms or conditions such as chronic pain, tremor, Parkinson's disease, epilepsy, urinary or fecal incontinence, sexual dysfunction, obesity, or gastroparesis. A medical device may deliver electrical stimulation therapy via one or more leads that include electrodes located proximate to target locations associated with the brain, the spinal cord, pelvic nerves, peripheral nerves, or the gastrointestinal tract of a patient. Stimulation proximate the spinal cord, proximate the sacral nerve, within the brain, and proximate peripheral nerves are often referred to as spinal cord stimulation (SCS), sacral neuromodulation (SNM), deep brain stimulation (DBS), and peripheral nerve stimulation (PNS), respectively.

Electrical stimulation may be delivered to a patient by the medical device in a train of electrical pulses, and parameters of the electrical pulses may include a frequency, an amplitude, a pulse width, and a pulse shape. An evoked compound action potential (ECAP) is synchronous firing of a population of neurons which occurs in response to the application of a stimulus including, in some cases, an electrical stimulus by a medical device. The ECAP may be detectable as being a separate event from the stimulus itself, and the ECAP may reveal characteristics of the effect of the stimulus on the nerve fibers.

### SUMMARY

In general, systems, devices, and techniques are described for determining stimulation parameters of stimulation pulses based on a latency determined from ECAP signals. ECAP signals (which may be referred to in the plurality as ECAPS) are a measure of neural recruitment because each ECAP signal represents the superposition of electrical potentials generated from a population of axons firing in response to an electrical stimulus (e.g., a stimulation pulse). Changes in a feature or characteristic (e.g., an amplitude of a portion of the signal, area under the curve of the signal, curve shape, timing of one or more peaks, etc.) of ECAP signals occur as a function of how many nerve fibers, and which type of fibers, have been activated by the delivered stimulation pulse. A system can use the ECAP signals for a variety of purposes, such as a closed-loop feedback variable to inform electrical stimulation therapy adjustments. However, the latency (e.g., the delay) of one or more features of the ECAP signal elicited by the electrical stimulus can depend on one or more stimulation parameter values that define the electrical stimulus.

As described herein, a system may determine a latency characteristic from one or more ECAP signals elicited by respective stimulation pulses. The latency characteristic may be representative of an ECAP signal elicited by a single stimulation pulse or representative of multiple ECAP signals elicited by stimulation pulses defined by different parameter values. In some examples, the system may cycle stimulation on and off based on the latency characteristic increasing or decreasing, or exceeding a latency threshold. The delivered stimulation therapy may include bursts of pulses that may be referred to as conditioning pulses and single stimulation pulses that elicit a detectable ECAP signal. The conditioning pulses may affect what types of fibers are activated by the single stimulation pulse that follows the burst of pulses. Therefore, in some examples, the system may adjust one or more stimulation parameters that define the burst of pulses in order to modulate fiber activation caused by a stimulation pulse to follow the burst of pulses.

In one example, a system includes system comprising processing circuitry configured to: control delivery of a first burst of pulses, each pulse of the first burst of pulses defined by a first parameter set; subsequent to delivery of the first burst of pulses, control delivery of a stimulation pulse defined by a second parameter set different from the first parameter set; receive information representative of an evoked compound action potential (ECAP) signal elicited by the stimulation pulse; determine an ECAP characteristic of the ECAP signal, the ECAP characteristic indicative of a latency of one of more nerve fibers activated by the stimulation pulse; determine, based on the ECAP characteristic, one or more parameter values of the first parameter set to generate a third parameter set for a second burst of pulses; and control delivery of the second burst of pulses according to the third parameter set.

In another example, a method includes controlling, by processing circuitry, delivery of a first burst of pulses, each pulse of the first burst of pulses defined by a first parameter set; subsequent to delivery of the first burst of pulses, controlling, by the processing circuitry, delivery of a stimulation pulse defined by a second parameter set different from the first parameter set; receiving, by the processing circuitry, information representative of an evoked compound action potential (ECAP) signal elicited by the stimulation pulse; determining, by the processing circuitry, an ECAP characteristic of the ECAP signal, the ECAP characteristic indicative of a latency of one of more nerve fibers activated by the stimulation pulse; determining, by the processing circuitry and based on the ECAP characteristic, one or more parameter values of the first parameter set to generate a third parameter set for a second burst of pulses; and controlling, by the processing circuitry, delivery of the second burst of pulses according to the third parameter set.

In another example, a computer-readable storage medium comprising instructions that, when executed by processing circuitry, cause the processing circuitry to control delivery of a first burst of pulses, each pulse of the first burst of pulses defined by a first parameter set; subsequent to delivery of the first burst of pulses, control delivery of a stimulation pulse defined by a second parameter set different from the first parameter set; receive information representative of an evoked compound action potential (ECAP) signal elicited by the stimulation pulse; determine an ECAP characteristic of the ECAP signal, the ECAP characteristic indicative of a latency of one of more nerve fibers activated by the stimulation pulse; determine, based on the ECAP characteristic, one or more parameter values of the first parameter set to generate a third parameter set for a second burst of pulses; and control delivery of the second burst of pulses according to the third parameter set.

In another example, a method comprises controlling delivery of a stimulation pulse via one or more electrodes; receiving information representative of an evoked compound action potential (ECAP) signal elicited by the stimulation pulse; determining an ECAP characteristic of the ECAP signal, the ECAP characteristic indicative of a latency of one of more nerve fibers activated by the stimulation pulse; and determining, based on the ECAP characteristic indicative of the latency, an implant location for the one or more electrodes.

The summary is intended to provide an overview of the subject matter described in this disclosure. It is not intended to provide an exclusive or exhaustive explanation of the systems, device, and methods described in detail within the accompanying drawings and description below. Further details of one or more examples of this disclosure are set forth in the accompanying drawings and in the description below. Other features, objects, and advantages of the techniques will be apparent from the description and drawings, and from the claims.

### BRIEF DESCRIPTION OF DRAWINGS

FIG. 1 is a conceptual diagram illustrating an example system that includes a medical device programmer and an IMD according to the techniques of the disclosure.

FIG. 2 is a block diagram of the example IMD of FIG. 1.

FIG. 3 is a block diagram of the example external programmer of FIG. 1.

FIG. 4A is a graph of an example ECAP signal sensed from a stimulation pulse.

FIGS. 4B and 4C are graphs of example ECAP signals sensed from stimulation pulses having different current amplitudes.

FIG. 5 is a timing diagram illustrating one example of electrical stimulation pulses and respective sensed ECAPs, in accordance with one or more techniques of this disclosure.

FIG. 6A is a graph of example differential ECAP curves indicative of different fiber latencies at different amplitude values.

FIG. 6B is a graph of differential ECAP characteristics representative of the differential ECAP curves in FIG. 6A.

FIG. 6C includes graphs of different ECAP characteristic values for different parameters defining burst of pulses and stimulation pulses.

FIGS. 6D and 6E are graphs of different nerve fiber locations with respect to the midline of the spinal cord.

FIGS. 6F and 6G are graphs representing different ECAP signals from different stimulation amplitudes.

FIGS. 6H and 6I are graphs representing different components of ECAP signals using a principle component analysis (PCA).

FIG. 6J provides graphs of correlation of different components of a PCA of ECAP signals.

FIG. 6K is a graph illustrating example latencies presented as theta of different ECAP signals and example indications that can be presented.

FIG. 6L provides graphs of example ECAP signals and theta values representative of latency for that ECAP signal.

FIG. 7A is a flow diagram illustrating an example technique for adjusting parameter values based on an indication of latency in ECAP signals.

FIG. 7B is a flow diagram illustrating an example technique for cycling the delivery of conditioning bursts of pulses while continuing to deliver stimulation pulses.

FIG. 7C is a flow diagram illustrating an example technique for determining differential latency characteristics from ECAP signals, in accordance with one or more techniques of this disclosure.

FIG. 8 is a diagram illustrating an example technique for adjusting electrical stimulation therapy.

FIG. 9 is a graph illustrating a relationship between sensed ECAP voltage amplitude and stimulation current amplitude, in accordance with one or more techniques of this disclosure.

Like reference characters denote like elements throughout the description and figures.

### DETAILED DESCRIPTION

The disclosure describes examples of medical devices, systems, and techniques for determining stimulation parameters based on latency characteristics derived from ECAP signals. Electrical stimulation therapy is typically delivered to a target tissue (e.g., nerves of the spinal cord or muscle) of a patient via two or more electrodes. Parameters of the electrical stimulation therapy (e.g., electrode combination, voltage or current amplitude, pulse width, pulse frequency, etc.) are selected by a clinician and/or the patient to provide relief from various symptoms, such as pain, nervous system disorders, muscle disorders, etc.

Typically, electrical stimulation therapy may be configured to cause paresthesia, which may include a tingling or buzzing feeling that may reduce pain perceived by the patient. Large nerve fibers being activated may be responsible for paresthesia. In some examples, high frequency, or high dose, stimulation may be delivered in an attempt to cause anesthesia, or a reduction in pain without paresthesia. Smaller nerve fibers being activated may cause this anesthesia affect. However, the high dose stimulation can cause adaptation in minutes or hours that reduces the anesthesia affect. Adaptation is a phenomenon for all fiber types, so it is unclear if high dose stimulation can maintain small fiber activation and the anesthesia affect over time. Moreover, there is no mechanism that can provide feedback on what types of fibers are being activated so that the system can adjust stimulation parameter values as needed to continue to target small fiber activation while reducing activation of large fibers that may cause paresthesia.

As described herein, a system may determine a latency characteristic from one or more ECAP signals elicited by respective stimulation pulses. Such a latency characteristic determined from ECAP signals may provide feedback about which types of nerve fibers are activated by the delivered stimulation. Larger nerve fibers may cause paresthesia when activated, and smaller fibers may cause anesthesia for the patient. Since ECAP responses may be faster for larger nerve fibers than smaller nerve fibers, the system can adjust stimulation parameters based on the latency characteristic in order to target larger or smaller nerve fibers to achieve a desired therapeutic effect, such as increased anesthesia and decreased paresthesia. The latency characteristic may be representative of an ECAP signal elicited by a single stimulation pulse or representative of multiple ECAP signals elicited by stimulation pulses defined by different parameter values. In some examples, the system may cycle stimulation (e.g., all stimulation pulses, bursts of pulses, and/or single stimulation pulses) on and off based on the latency characteristic increasing or decreasing, or exceeding a latency threshold.

The system may deliver stimulation therapy that includes bursts of pulses that may be referred to as conditioning pulses and single stimulation pulses that follow the burst of pulses and elicit a detectable ECAP signal. The conditioning pulses may affect what types of fibers are activated by the single stimulation pulse that follows the burst of pulses. For example, the conditioning pulses may be targeted to larger nerve fibers in order to suppress large fiber activation from the following stimulation pulse. The conditioning pulses may have an sub-perception threshold intensity (amplitude and/or pulse width) selected such that the patient does not perceive delivery of the conditioning pulses. Therefore, in some examples, the system may adjust one or more stimulation parameters, based on the latency characteristic, that define the burst of pulses in order to modulate fiber activation caused by a stimulation pulse to follow the burst of pulses. The resulting stimulation pulse may thus activate a larger portion of smaller fibers associated with anesthesia and a relatively smaller portion of larger fibers associated with anesthesia. Although a goal of the system may be to only activate smaller fibers instead of larger fibers to achieve only anesthetic stimulation, the system may achieve a ratio of small fiber activation to large fiber activation that is high enough to provide an anesthesia response greater than any remaining paresthesia effect. Bursts of pulses described herein may be a plurality of pulses delivered as a pre-programmed number of consecutive pulses or a pre-programmed duration of time in which the pulses are delivered at the predetermined inter-pulse frequency.

Effective stimulation therapy may also rely on a certain level of neural recruitment at a target nerve or group of nerve fibers (e.g., at a target ECAP characteristic value or below a threshold ECAP characteristic value). This effective stimulation therapy may provide relief from one or more conditions (e.g., patient perceived pain) without an unacceptable level of side effects (e.g., overwhelming perception of stimulation). In order to maintain effective stimulation therapy as the patient moves or therapy progresses over time, a system may also use the characteristic value of an ECAP signal as feedback for adjusting a stimulation parameter (e.g., increase or decrease the stimulation parameter value) to increase or decrease the neural recruitment back to the neural recruitment associated with effective stimulation therapy.

However, the patient may have different sensitivities to increasing stimulation intensity and decreasing stimulation intensity. For example, the patient may be more sensitive to increasing stimulation intensity (e.g., increasing a current amplitude value in a subsequent pulse) than decreasing stimulation intensity (e.g., decreasing a current amplitude value in a subsequent pulse). Without tailoring changes to stimulation parameter values to account for increasing or decreasing stimulation intensity of stimulation pulses, the system may not efficiently achieve the desired neural recruitment levels for the patient (e.g., cause patient discomfort or reduce therapy efficacy). Therefore, a system may employ different gain values for increasing stimulation intensity and decreasing stimulation intensity, as determined by the difference between a target ECAP characteristic value and the detected ECAP characteristic value (e.g., an ECAP differential value).

If the patient changes posture or otherwise engages in physical activity, the distance between the electrodes and the nerve changes as well. This change in distance can cause loss of effective therapy and/or side effects if the parameter values that define stimulation are not adjusted to compensate for the change in distance. The different distance between electrodes and the target nerve (e.g., caused by a shift from one posture state to another) may also result in different sensitivities to stimulation intensity (e.g., smaller distances may result in greater sensitivities to changes in stimulation intensity). For example, the changing distance may change the efficacy of conditioning pulses and/or the stimulation pulse to provide effective anesthesia and reduce paresthesia. If a system does not adjust the control policy for these changes, adjustments to stimulation parameter values may not be sufficient to maintain effective therapy or may provide stimulation that is too strong at that posture state. Therefore, it may be beneficial to maintain effective therapy by the system adjusting how stimulation intensity is changed within a given posture state and/or changing target ECAP characteristic values when a posture state of the patient has changed. A system may thus employ a closed-loop control system for adjusting parameter values of electrical stimulation pulses based on the features of sensed ECAP signals. In some examples, the system may modulate one or more stimulation parameter values based on one of or more ECAP characteristics indicative of a latency of the ECAP signal and the amplitude of the ECAP signal.

The systems and techniques may provide one or more advantages over other types of therapy. For example, the system may automatically (e.g., in a closed-loop control policy) adjust one or more stimulation parameter values based on a latency characteristic in order to increase the ratio of small fiber activation to large fiber activation and increase an anesthesia effect of the stimulation therapy. In some examples, the system may achieve stimulation therapy that only provides an anesthesia affect with no paresthesia affect. In addition, the system may cycle stimulation pulses (e.g., the conditioning pulses and/or the stimulation pulses) by monitoring the latency characteristic and maintaining the latency of the ECAP signals exceeding a latency threshold. This cycling may reduce power consumption of the system and/or reduce adaptation of the nerve fibers that may reduce therapy efficacy over time.

Although electrical stimulation is generally described herein in the form of electrical stimulation pulses, electrical stimulation may be delivered in non-pulse form in other examples. For example, electrical stimulation may be delivered as a signal having various waveform shapes, frequencies, and amplitudes. Therefore, electrical stimulation in the form of a non-pulse signal may be a continuous signal than may have a sinusoidal waveform or other continuous waveform.

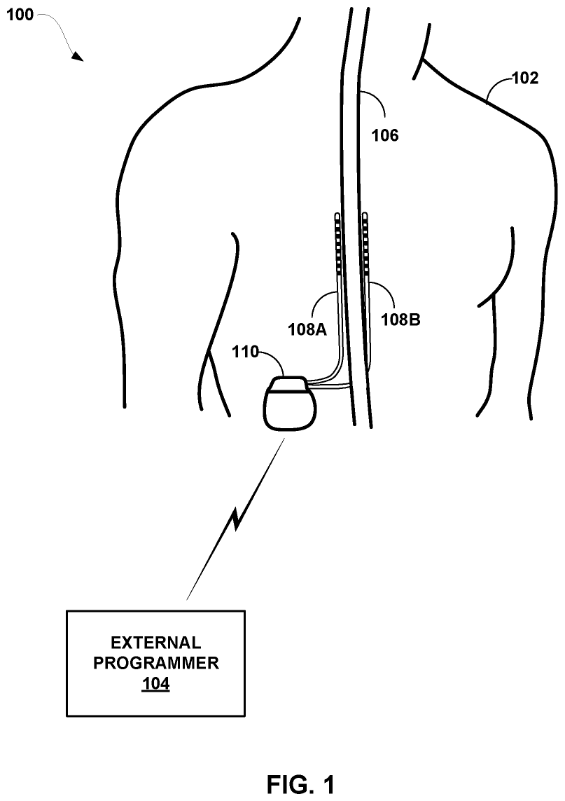

FIG. 1 is a conceptual diagram illustrating example system 100 that includes implantable medical device (IMD) 110 to deliver electrical stimulation therapy to patient 102. Although the techniques described in this disclosure are generally applicable to a variety of medical devices including external devices and IMDs, application of such techniques to IMDs and, more particularly, implantable electrical stimulators (e.g., neurostimulators) will be described for purposes of illustration. More particularly, the disclosure will refer to an implantable SCS system for purposes of illustration, but without limitation as to other types of medical devices or other therapeutic applications of medical devices.

As shown in FIG. 1, system 100 includes an IMD 110, leads 108A and 108B, and external programmer 104 shown in conjunction with a patient 102, who is ordinarily a human patient. In the example of FIG. 1, IMD 110 is an implantable electrical stimulator that is configured to generate and deliver electrical stimulation therapy to patient 102 via one or more electrodes of a plurality of electrodes carried by leads 108A and/or 108B (collectively, “leads 108”), e.g., for relief of chronic pain or other symptoms. In other examples, IMD 110 may be coupled to a single lead carrying multiple electrodes or more than two leads each carrying multiple electrodes. In some examples, the stimulation signals, or pulses, may be configured to elicit detectable ECAP signals that IMD 110 may use to determine the posture state occupied by patient 102 and/or determine how to adjust one or more parameters that define stimulation therapy. IMD 110 may be a chronic electrical stimulator that remains implanted within patient 102 for weeks, months, or even years. In other examples, IMD 110 may be a temporary, or trial, stimulator used to screen or evaluate the efficacy of electrical stimulation for chronic therapy. In one example, IMD 110 is implanted within patient 102, while in another example, IMD 110 is an external device coupled to percutaneously implanted leads. In some examples, IMD 110 uses one or more leads, while in other examples, IMD 110 is leadless.

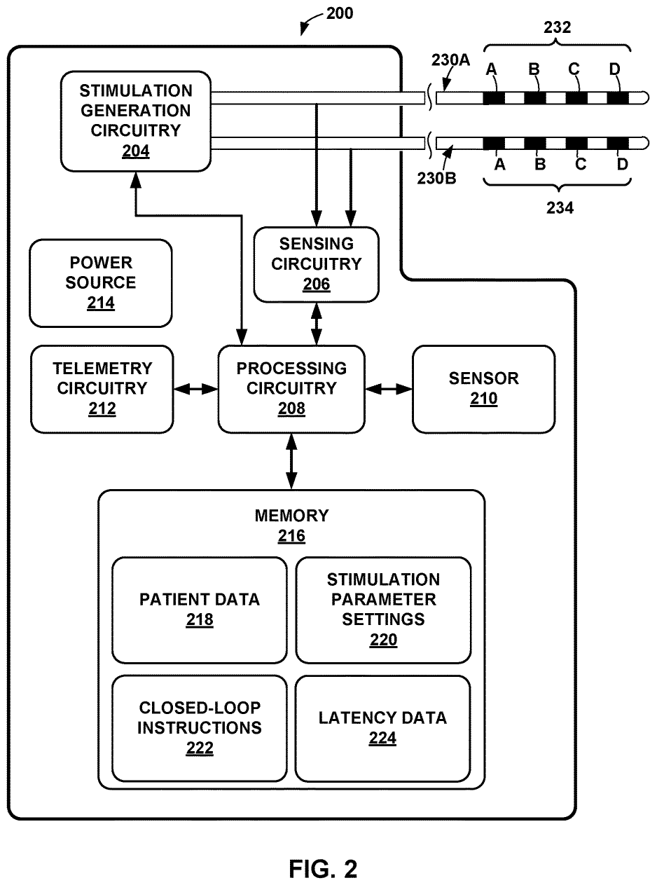

IMD 110 may be constructed of any polymer, metal, or composite material sufficient to house the components of IMD 110 (e.g., components illustrated in FIG. 2) within patient 102. In this example, IMD 110 may be constructed with a biocompatible housing, such as titanium or stainless steel, or a polymeric material such as silicone, polyurethane, or a liquid crystal polymer, and surgically implanted at a site in patient 102 near the pelvis, abdomen, or buttocks. In other examples, IMD 110 may be implanted within other suitable sites within patient 102, which may depend, for example, on the target site within patient 102 for the delivery of electrical stimulation therapy. The outer housing of IMD 110 may be configured to provide a hermetic seal for components, such as a rechargeable or non-rechargeable power source. In addition, in some examples, the outer housing of IMD 110 is selected from a material that facilitates receiving energy to charge the rechargeable power source.

Electrical stimulation energy, which may be constant current or constant voltage-based pulses, for example, is delivered from IMD 110 to one or more target tissue sites of patient 102 via one or more electrodes (not shown) of implantable leads 108. In the example of FIG. 1, leads 108 carry electrodes that are placed adjacent to the target tissue of spinal cord 106. One or more of the electrodes may be disposed at a distal tip of a lead 108 and/or at other positions at intermediate points along the lead. Leads 108 may be implanted and coupled to IMD 110. The electrodes may transfer electrical stimulation generated by an electrical stimulation generator in IMD 110 to tissue of patient 102. Although leads 108 may each be a single lead, lead 108 may include a lead extension or other segments that may aid in implantation or positioning of lead 108. In some other examples, IMD 110 may be a leadless stimulator with one or more arrays of electrodes arranged on a housing of the stimulator rather than leads that extend from the housing. In addition, in some other examples, system 100 may include one lead or more than two leads, each coupled to IMD 110 and directed to similar or different target tissue sites.

The electrodes of leads 108 may be electrode pads on a paddle lead, circular (e.g., ring) electrodes surrounding the body of the lead, conformable electrodes, cuff electrodes, segmented electrodes (e.g., electrodes disposed at different circumferential positions around the lead instead of a continuous ring electrode), any combination thereof (e.g., ring electrodes and segmented electrodes) or any other type of electrodes capable of forming unipolar, bipolar or multipolar electrode combinations for therapy. Ring electrodes arranged at different axial positions at the distal ends of lead 108 will be described for purposes of illustration.

The deployment of electrodes via leads 108 is described for purposes of illustration, but arrays of electrodes may be deployed in different ways. For example, a housing associated with a leadless stimulator may carry arrays of electrodes, e.g., rows and/or columns (or other patterns), to which shifting operations may be applied. Such electrodes may be arranged as surface electrodes, ring electrodes, or protrusions. As a further alternative, electrode arrays may be formed by rows and/or columns of electrodes on one or more paddle leads. In some examples, electrode arrays include electrode segments, which are arranged at respective positions around a periphery of a lead, e.g., arranged in the form of one or more segmented rings around a circumference of a cylindrical lead. In other examples, one or more of leads 108 are linear leads having 8 ring electrodes along the axial length of the lead. In another example, the electrodes are segmented rings arranged in a linear fashion along the axial length of the lead and at the periphery of the lead.

The stimulation parameter set of a therapy stimulation program that defines the stimulation pulses of electrical stimulation therapy by IMD 110 through the electrodes of leads 108 may include information identifying which electrodes have been selected for delivery of stimulation according to a stimulation program, the polarities of the selected electrodes, i.e., the electrode combination for the program, voltage or current amplitude, pulse frequency, pulse width, pulse shape of stimulation delivered by the electrodes. These stimulation parameters values that make up the stimulation parameter set that defines pulses may be predetermined parameter values defined by a user and/or automatically determined by system 100 based on one or more factors or user input.

Although FIG. 1 is directed to SCS therapy, e.g., used to treat pain, in other examples system 100 may be configured to treat any other condition that may benefit from electrical stimulation therapy. For example, system 100 may be used to treat tremor, Parkinson's disease, epilepsy, a pelvic floor disorder (e.g., urinary incontinence or other bladder dysfunction, fecal incontinence, pelvic pain, bowel dysfunction, or sexual dysfunction), obesity, gastroparesis, or psychiatric disorders (e.g., depression, mania, obsessive compulsive disorder, anxiety disorders, and the like). In this manner, system 100 may be configured to provide therapy taking the form of deep brain stimulation (DBS), peripheral nerve stimulation (PNS), peripheral nerve field stimulation (PNFS), cortical stimulation (CS), pelvic floor stimulation, gastrointestinal stimulation, or any other stimulation therapy capable of treating a condition of patient 102.

In some examples, leads 108 includes one or more sensors configured to allow IMD 110 to monitor one or more parameters of patient 102, such as patient activity, pressure, temperature, or other characteristics. The one or more sensors may be provided in addition to, or in place of, therapy delivery by lead 108.

IMD 110 is generally configured to deliver electrical stimulation therapy to patient 102 via selected combinations of electrodes carried by one or both of leads 108, alone or in combination with an electrode carried by or defined by an outer housing of IMD 110. The target tissue for the electrical stimulation therapy may be any tissue affected by electrical stimulation, which may be in the form of electrical stimulation pulses or continuous waveforms. In some examples, the target tissue includes nerves, smooth muscle, or skeletal muscle. In the example illustrated by FIG. 1, the target tissue is tissue proximate spinal cord 106, such as within an intrathecal space or epidural space of spinal cord 106, or, in some examples, adjacent nerves that branch off spinal cord 106. Leads 108 may be introduced into spinal cord 106 in via any suitable region, such as the thoracic, cervical, or lumbar regions. Stimulation of spinal cord 106 may, for example, prevent pain signals from traveling through spinal cord 106 and to the brain of patient 102. Patient 102 may perceive the interruption of pain signals as a reduction in pain and, therefore, efficacious therapy results. In other examples, stimulation of spinal cord 106 may produce paresthesia which may be reduce the perception of pain by patient 102, and thus, provide efficacious therapy results.

IMD 110 is configured to generate and deliver electrical stimulation therapy to a target stimulation site within patient 102 via the electrodes of leads 108 to patient 102 according to one or more therapy stimulation programs. A therapy stimulation program defines values for one or more parameters (e.g., a parameter set) that define an aspect of the therapy delivered by IMD 110 according to that program. For example, a therapy stimulation program that controls delivery of stimulation by IMD 110 in the form of pulses may define values for voltage or current pulse amplitude, pulse width, pulse rate (e.g., pulse frequency), burst frequency, burst length, a number of pulses within a burst of pulses, electrode combination, pulse shape, etc. for stimulation pulses delivered by IMD 110 according to that program.

A user, such as a clinician or patient 102, may interact with a user interface of an external programmer 104 to program IMD 110. Programming of IMD 110 may refer generally to the generation and transfer of commands, programs, or other information to control the operation of IMD 110. In this manner, IMD 110 may receive the transferred commands and programs from external programmer 104 to control stimulation, such as stimulation pulses that provide electrical stimulation therapy. For example, external programmer 104 may transmit therapy stimulation programs, stimulation parameter adjustments, therapy stimulation program selections, posture states, user input, or other information to control the operation of IMD 110, e.g., by wireless telemetry or wired connection.

In some cases, external programmer 104 may be characterized as a physician or clinician programmer if it is primarily intended for use by a physician or clinician. In other cases, external programmer 104 may be characterized as a patient programmer if it is primarily intended for use by a patient. A patient programmer may be generally accessible to patient 102 and, in many cases, may be a portable device that may accompany patient 102 throughout the patient's daily routine. For example, a patient programmer may receive input from patient 102 when the patient wishes to terminate or change electrical stimulation therapy, or when a patient perceives stimulation being delivered. In general, a physician or clinician programmer may support selection and generation of programs by a clinician for use by IMD 110, whereas a patient programmer may support adjustment and selection of such programs by a patient during ordinary use. In other examples, external programmer 104 may include, or be part of, an external charging device that recharges a power source of IMD 110. In this manner, a user may program and charge IMD 110 using one device, or multiple devices.

As described herein, information may be transmitted between external programmer 104 and IMD 110. Therefore, IMD 110 and external programmer 104 may communicate via wireless communication using any techniques known in the art. Examples of communication techniques may include, for example, radiofrequency (RF) telemetry and inductive coupling, but other techniques are also contemplated. In some examples, external programmer 104 includes a communication head that may be placed proximate to the patient's body near the IMD 110 implant site to improve the quality or security of communication between IMD 110 and external programmer 104. Communication between external programmer 104 and IMD 110 may occur during power transmission or separate from power transmission.

In some examples, IMD 110, in response to commands from external programmer 104, delivers electrical stimulation therapy according to a plurality of therapy stimulation programs to a target tissue site of the spinal cord 106 of patient 102 via electrodes (not depicted) on leads 108. In some examples, IMD 110 modifies therapy stimulation programs as therapy needs of patient 102 evolve over time. For example, the modification of the therapy stimulation programs may cause the adjustment of at least one parameter of the plurality of stimulation pulses. When patient 102 receives the same therapy for an extended period, the efficacy of the therapy may be reduced. In some cases, parameters of the plurality of stimulation pulses may be automatically updated.

As described herein, IMD 110 may be configured to detect ECAP signals which are representative of the number of nerve fibers, and the types of fibers (e.g., size of fibers which may correlated to propagation speed), activated by a delivered stimulation signal (e.g., a delivered pulse). As the patient moves, the distance between the electrodes and the target tissues changes. Since neural recruitment at the nerves is a function of stimulation intensity (e.g., amplitude and/or pulse frequency) and distance between the target tissue and the electrodes, movement of the electrode closer to the target tissue may result in increased neural recruitment (e.g., possible painful sensations or adverse motor function), and movement of the electrode further from the target tissue may result in decreased efficacy of the therapy for the patient. Certain patient postures (which may or may not include patient activity) may be representative of respective distances (or changes in distance) between electrodes and nerves and thus be an informative feedback variable for modulating stimulation therapy.

In some examples, a patient may experience discomfort or pain caused by transient patient conditions, which is referred to herein as transient overstimulation. The electrodes can move closer to the target tissue for a number of reasons including coughing, sneezing, laughing, valsalva maneuvers, leg lifting, cervical motions, deep breathing, or another transient patient movement. If a system is delivering stimulation during these movements, the patient may perceive the stimulation as stronger (and possibly uncomfortable) due to the decreased distance between electrodes and target tissue in a short amount of time. Although a patient may anticipate such movements and preemptively reduce stimulation intensity in an attempt to avoid these uncomfortable sensations, these patient actions interfere with normal activities and may not be sufficient to avoid uncomfortable stimulation at all times. In addition, changing distances between the electrodes and nerves may result in ineffective conditioning pulses that do not suppress larger fibers effectively as described herein.

ECAPs are a measure of neural recruitment because each ECAP signal represents the superposition of electrical potentials generated from a population of axons firing in response to an electrical stimulus (e.g., a stimulation pulse). Changes in a feature or characteristic (e.g., an amplitude of a portion of the signal or area under the curve of the signal) of ECAP signals occur as a function of how many axons have been activated by the delivered stimulation pulse. For a given set of parameter values that define the stimulation pulse and a given distance between the electrodes and target nerve, the detected ECAP signal may have a certain characteristic value (e.g., amplitude, or area under a curve). Therefore, a system can detect one or more features of a sensed ECAP signal and determine that the distance between electrodes and nerves has increased or decreased in response to determining that the feature (or measured ECAP characteristic value based on one or more features) has increased or decreased. For example, if the set of parameter values stays the same and the ECAP characteristic value of amplitude increases, the system can determine that the distance between electrodes and the nerve has decreased. In addition, the latency, or change in time, of one or more peaks in the ECAP signal may be indicative of the types of fibers activated by the delivered stimulation pulse.

The ECAP signal may include several features such as different peaks that include a first peak (P1), a trough (N1), a second peak (P2), and sometimes additional troughs and peaks. The system may determine an ECAP characteristic value for the ECAP signal based on one or more of these features (e.g., the absolute amplitude between the N1 and P2 features). These amplitude related features of the ECAP signal may be referred to as an ECAP characteristic amplitude. However, the detection of one or more features of the ECAP signal may be difficult as features of the ECAP signal may change in time from delivery of the stimulation pulse that elicits the ECAP signal. This change in the delay of the ECAP features from the delivered stimulation pulse can be referred to as latency. A change in latency of the features in the ECAP signal may be due to different types of nerve fibers being activated from differences in the parameter values of the stimulation pulse. This latency characteristic may also be a type of ECAP characteristic. A system may employ sensing windows to identify respective features in the ECAP signal, but if a change in latency causes a feature to occur outside for the sensing window, the system may not identify the appropriate feature of the ECAP signal. A consequence of failing to identify the appropriate feature in the ECAP signal may result in an inability of the system to appropriately adjust electrical stimulation therapy based on the sensed ECAP signals.

IMD 110 may employ various techniques in order to appropriately detect the ECAP signal or one or more features of the ECAP signal that is elicited by the delivered stimulation pulse. The ECAP signal may include several features such as different peaks that include a first peak (P1), a trough (N1), a second peak (P2), and additional troughs and peaks in some situations. The detection of one or more features of the ECAP signal may be difficult as features of the ECAP signal may change in time with respect to the delivery of the stimulation pulse that elicits the ECAP signal. This change in the delay of the ECAP features from the delivered stimulation pulse can be referred to as latency. A change in latency of the features in the ECAP signal may be due to different types of nerve fibers being activated from differences in the parameter values of the stimulation pulse. For example, changing a parameter value for a stimulation pulse may change the ratio of slow nerve fibers to fast nerve fibers. Other factors may also cause the latency to be different for different parameter values. In some examples, IMD 110 may employ one or more sensing windows to identify respective features in each ECAP signal, which IMD 110 may change over time. In some examples, IMD 110 may determine the latency characteristic based on a differential of the amplitudes of different peaks in the ECAP signal.

Efficacy of electrical stimulation therapy (e.g., neural recruitment) may be indicated by one or more characteristics (e.g., an amplitude of or between one or more peaks or an area under the curve of one or more peaks) of an action potential that is evoked by a stimulation pulse delivered by IMD 110 (i.e., a characteristic value of the ECAP signal). Electrical stimulation therapy delivery by leads 108 of IMD 110 may cause neurons within the target tissue to evoke a compound action potential that travels up and down the target tissue, eventually arriving at sensing electrodes of IMD 110. Furthermore, stimulation may also elicit at least one ECAP signal, and ECAPs responsive to stimulation may also be a surrogate for the effectiveness of the therapy. The amount of action potentials (e.g., number of neurons propagating action potential signals) that are evoked may be based on the various parameters of electrical stimulation pulses such as amplitude, pulse width, frequency, pulse shape (e.g., slew rate at the beginning and/or end of the pulse), etc. In addition, the amount of action potentials that are evoked may vary depending on whether the intensity of stimulation pulses is increasing or decreasing from successive pulses. The slew rate may define the rate of change of the voltage and/or current amplitude of the pulse at the beginning and/or end of each pulse or each phase within the pulse. For example, a very high slew rate indicates a steep or even near vertical edge of the pulse, and a low slew rate indicates a longer ramp up (or ramp down) in the amplitude of the pulse. In some examples, these parameters contribute to an intensity of the electrical stimulation. In addition, a characteristic of the ECAP signal (e.g., an amplitude) may change based on the distance between the stimulation electrodes and the nerves subject to the electrical field produced by the delivered stimulation pulses.

Some example techniques for adjusting stimulation parameter values for stimulation pulses (e.g., pulses that may or may not contribute to therapy for the patient) are based on comparing the value of a characteristic of a measured ECAP signal to a target ECAP characteristic value. In response to delivering a stimulation pulse defined by a set of stimulation parameter values, IMD 110, via two or more electrodes interposed on leads 108, senses electrical potentials of tissue of the spinal cord 106 of patient 102 to measure the electrical activity of the tissue. IMD 110 senses ECAPs from the target tissue of patient 102, e.g., with electrodes on one or more leads 108 and associated sense circuitry. In some examples, IMD 110 receives a signal indicative of the ECAP from one or more sensors, e.g., one or more electrodes and circuitry, internal or external to patient 102. Such an example signal may include a signal indicating an ECAP of the tissue of patient 102. Examples of the one or more sensors include one or more sensors configured to measure a compound action potential of patient 102, or a physiological effect indicative of a compound action potential. For example, to measure a physiological effect of a compound action potential, the one or more sensors may be an accelerometer, a pressure sensor, a bending sensor, a sensor configured to detect a posture of patient 102, or a sensor configured to detect a respiratory function of patient 102. However, in other examples, external programmer 104 receives a signal indicating a compound action potential in the target tissue of patient 102 and transmits a notification to IMD 110.

In the example of FIG. 1, IMD 110 is described as performing a plurality of processing and computing functions. However, external programmer 104 instead may perform one, several, or all of these functions. In this alternative example, IMD 110 functions to relay sensed signals to external programmer 104 for analysis, and external programmer 104 transmits instructions to IMD 110 to adjust the one or more parameters defining the electrical stimulation therapy based on analysis of the sensed signals. For example, IMD 110 may relay the sensed signal indicative of an ECAP to external programmer 104. External programmer 104 may compare the parameter value of the ECAP to the target ECAP characteristic value, and in response to the comparison, external programmer 104 may instruct IMD 110 to adjust one or more stimulation parameter that defines the electrical stimulation informed pulses and, in some examples, control pulses, delivered to patient 102. In some examples, the system changes the target ECAP characteristic value and/or growth rate(s) over a period of time, such as according to a change to a stimulation threshold (e.g., a perception threshold or detection threshold specific for the patient). The system may be programmed to change the target ECAP characteristic in order to adjust the intensity of informed pulses to provide varying sensations to the patient (e.g., increase or decrease the volume of neural activation). Although the system may change the target ECAP characteristic value, received ECAP signals may still be used by the system to adjust one or more parameter values of the informed pulses and/or control pulses in order to meet the target ECAP characteristic value.

One or more devices within system 100, such as IMD 110 and/or external programmer 104, may perform various functions as described herein. For example, IMD 110 may include stimulation circuitry configured to deliver electrical stimulation, sensing circuitry configured to sense a plurality ECAP signals, and processing circuitry. The processing circuitry may be configured to control the stimulation circuitry to deliver a plurality of electrical stimulation pulses having different amplitude values and control the sensing circuitry to detect, after delivery of each electrical stimulation pulse of the plurality of electrical stimulation pulses, a respective ECAP signal of the plurality of ECAP signals.

IMD 110 may modulate or adjust one or more stimulation parameters that at least partially define electrical stimulation, and IMD 110 may adjust the one or more stimulation parameters based on whether or not stimulation intensity is to be increased or decreased, and in some examples, also based on a detected posture state of the patient 102. For example, IMD 110 may select a gain value according to whether or not IMD 110 needs to increase or decrease stimulation intensity according to the detected ECAP characteristic value (e.g., an ECAP differential value indicating a positive or negative relationship between the detected ECAP characteristic value and a target ECAP characteristic value). IMD 110 may also use the detected posture state to determine how to employ ECAP signals in a closed-loop feedback system for adjusting stimulation parameters. In one example, IMD 110 includes stimulation generation circuitry configured to generate and deliver electrical stimulation to patient 102 according to one or more sets of stimulation parameters that at least partially define the pulses of the electrical stimulation. Each set of stimulation parameters may include at least one of an amplitude, a pulse width, a pulse frequency, or a pulse shape.

IMD 110 may include sensing circuitry configured to sense an ECAP signal elicited by delivered electrical stimulation, such as a stimulation pulse. IMD 110 may also include processing circuitry configured to control stimulation circuitry to deliver a first electrical stimulation pulse to patient 102 according to a first value of a stimulation parameter and determine a characteristic value of the ECAP signal elicited from the electrical stimulation. IMD 110 may then determine an ECAP differential value that indicates whether the characteristic value of the ECAP signal elicited by the first electrical stimulation pulse is one of greater than or equal to a selected ECAP characteristic value or less than the selected ECAP characteristic value. For example, IMD 110 may compare the characteristic value of the ECAP signal to the selected ECAP characteristic value, and the comparison may indicate whether IMD 110 may need to increase or decrease stimulation intensity of stimulation pulses in order to achieve the selected ECAP characteristic value (e.g., a target ECAP characteristic value).

In other examples, IMD 110 may not attempt to maintain consistent nerve activation by modulating stimulation pulses to achieve a target ECAP characteristic value. Instead, IMD 110 may monitor characteristic values of ECAP signals and only take action when the characteristic value exceeds a threshold ECAP characteristic value. Characteristic values exceeding the threshold ECAP characteristic values may be indicative of increased stimulation perception that may be above an uncomfortable threshold or pain threshold for the patient. Therefore, reducing stimulation pulse intensity when the characteristic value exceeds this level of stimulation may reduce the likelihood that patient 102 experiences any uncomfortable sensations that may occur as a result of posture state changes or any transient movement. For example, IMD 110 may be configured to compare the characteristic value of the ECAP signal to a threshold ECAP characteristic value and determine that the characteristic value of the ECAP signal is greater than the threshold ECAP characteristic value (e.g., a positive ECAP differential value). Responsive to determining that the characteristic value of the ECAP signal is greater than the threshold ECAP characteristic value, IMD 110 may be configured to decrease the first value to the second value for the stimulation parameter of a subsequent stimulation pulse. As discussed above, IMD 110 may apply a gain value that is associated with the positive ECAP differential value or a negative ECAP differential value.

IMD 110 may continue to decrease the stimulation parameter value as long as the ECAP characteristic value continues to exceed the threshold ECAP characteristic value. Once, the stimulation parameter has been decreased, IMD 110 may attempt to increase the stimulation parameter value again back up to the predetermined first value intended for the stimulation pulses. IMD 110 may be configured to determine other characteristic values of subsequent ECAP signals elicited from electrical stimulation pulses delivered after sensing the first ECAP signal. In response to determining that another characteristic value of the subsequent ECAP signals decreases below the threshold ECAP characteristic value, IMD 110 may then increase the value of the stimulation parameter back up to a value limited to be less than or equal to the first value (e.g., back up to the predetermined value for stimulation pulses that may be determined by a set of stimulation parameters or therapy program). IMD 110 may use a different gain value to increase the stimulation parameter than the gain value used to decrease the stimulation parameter. In some examples, IMD 110 may iteratively increase the stimulation parameter value until the first value, or original value, is again reached after the characteristic values of the ECAP signal remain below the threshold ECAP characteristic value. IMD 110 may increase the stimulation parameter values at a slower rate than the stimulation parameter values are decreased, but, in other examples, IMD 110 may increase and decrease the stimulation parameters at the same rates.

During delivery of an electrical stimulation signal, IMD 110, via two or more electrodes interposed on leads 108, senses electrical potentials of tissue of the spinal cord 106 of patient 102 to measure the electrical activity of the tissue. IMD 110 senses ECAPs from the target tissue of patient 102, e.g., with electrodes on one or more leads 108 and associated sensing circuitry. In some examples, IMD 110 receives a signal indicative of the ECAP from one or more sensors, e.g., one or more electrodes and circuitry, internal or external to patient 102. Such an example signal may include a signal indicating an ECAP of the tissue of the patient 102. Examples of the one or more sensors include one or more sensors can measure a compound action potential of the patient 102, or a physiological effect indicative of a compound action potential. For example, to measure a physiological effect of a compound action potential, the one or more sensors may be an accelerometer, a pressure sensor, a bending sensor, a sensor can detect a posture of patient 102, or a sensor can detect a respiratory function of patient 102. However, in other examples, external programmer 104 receives a signal indicating a compound action potential in the target tissue of patient 102 and transmits a notification to IMD 110.

In the example of FIG. 1, IMD 110 described as performing a plurality of processing and computing functions. However, external programmer 104 instead may perform one, several, or all of these functions. In this alternative example, IMD 110 functions to relay sensed signals to external programmer 104 for analysis, and external programmer 104 transmits instructions to IMD 110 to adjust the one or more parameters defining the electrical stimulation signal based on analysis of the sensed signals. For example, IMD 110 may relay the sensed signal indicative of an ECAP to external programmer 104. External programmer 104 may compare the parameter value of the ECAP to the target ECAP characteristic value, and in response to the comparison, external programmer 104 may instruct IMD 110 to adjust one or more parameters that define the electrical stimulation signal.

In the example techniques described herein, the stimulation parameter values, latency curves, and the target ECAP characteristic values (e.g., values of the ECAP indicative of target stimulation intensity) may be initially set at the clinic but may be set and/or adjusted at home by patient 102. Once the target ECAP characteristic values are set, example techniques enable automatic adjustment of stimulation parameters to maintain consistent volume of neural activation and consistent therapy efficacy for the patient when the electrode-to-neuron distance changes and/or the types of nerve fibers activated changes. The ability to change the stimulation parameter values may also allow the therapy to have long term efficacy, with the ability to keep the intensity of the stimulation (e.g., as indicated by the ECAP) consistent by comparing the measured ECAP values to the target ECAP characteristic value. IMD 110 may perform these changes without intervention by a physician or patient 102.

In some examples, IMD 110 may not be able to measure ECAPs from stimulation that has certain pulse widths and/or pulse frequencies. For example, longer pulse widths and higher pulse frequencies (e.g., during a burst of pulses with a relatively high frequency) may result in a delivered stimulation pulse overlapping with an ECAP. Since the ECAP amplitude can be much lower amplitude than the stimulation pulse, the stimulation pulse(s) can cover up any ECAP characteristic value of the signal. For example, the system may not be able to detect ECAP signals between bursts of conditioning pulses as described herein. However, IMD 110 may use measured ECAPs elicited by stimulation pulses having shorter pulse widths and/or lower pulse frequencies to identify a combination of stimulation parameter values that produce an ECAP characteristic value (e.g., intensity) that is representative of effective therapy or other nerve characteristics, such as conditioning. IMD 110 may then select the parameters values for the pulses for which ECAP signals cannot properly be detected, such as the values of conditioning pulses which may be provided as bursts of pulses. In this manner, IMD 110 may measure ECAP signals elicited by some stimulation pulses (e.g., control pulses) and use the ECAP characteristic value derived thereof to inform adjustments to subsequent stimulation pulses such as conditioning pulses (e.g., informed pulses) that have different stimulation parameters. The informed pulses may be configured to produce a therapeutic effect, such as conditioning larger nerve fibers. The control pulses may, in some examples, produce a therapeutic effect such as anesthesia by causing smaller fibers to be activated. In some examples, the control pulses may have the same or longer pulse width than the informed pulses.

Although in one example IMD 110 takes the form of an SCS device, in other examples, IMD 110 takes the form of any combination of deep brain stimulation (DBS) devices, implantable cardioverter defibrillators (ICDs), pacemakers, cardiac resynchronization therapy devices (CRT-Ds), left ventricular assist devices (LVADs), implantable sensors, orthopedic devices, or drug pumps, as examples.

As described herein, system 100 can operate to perform any of the functionality described herein. For example, system 100 may include processing circuitry configured to control delivery of a first burst of pulses, where each pulse of the first burst of pulses defined by a first parameter set, and, subsequent to delivery of the first burst of pulses, control delivery of a stimulation pulse defined by a second parameter set different from the first parameter set. These bursts of pulses may be described as conditioning pulses in some examples, and the stimulation pulse may be configured to be delivered to target tissue which include at least some fibers conditioned by the burst of pulses.

System 100 can then receive information representative of an evoked compound action potential (ECAP) signal elicited by the stimulation pulse. In some examples, system 100 may include sensing circuitry configured to sense the ECAP signal and generate the information that can be received by processing circuitry of system 100. The processing circuitry can then determine an ECAP characteristic of the ECAP signal, the ECAP characteristic indicative of a latency of one of more nerve fibers activated by the stimulation pulse. In some examples, this ECAP characteristic indicative of latency may be referred to as a latency characteristic. The processing circuitry can then determine, based on the ECAP characteristic, one or more parameter values of the first parameter set to generate a third parameter set for a second burst of pulses and control delivery of the second burst of pulses according to the third parameter set.

In some examples, the processing circuitry may continue this process over time and compare the latencies indicated by different ECAP characteristics and adjust one or more parameter values defining the burst of pulses based on the change in latency. For example, the processing circuitry may be configured to receive a first ECAP characteristic indicative of a first latency of the one or more nerve fibers activated by delivery of the first stimulation pulse prior to the second stimulation pulse, where the processing circuitry is configured to determine the one or more parameters of the first parameter set by at least determining, based on a comparison of the first latency to the second latency, the one or more parameters of the first parameter set that defines the burst of pulses.

In this manner, the processing circuitry can attempt to achieve longer latencies indicative of a higher ratio of small fibers than large fibers that can result in a stronger anesthesia affect. In some examples, the processing circuitry is configured to determine that the second latency is less than the first latency, such as when the latency is decreasing over time. In response to determining that the second latency is less than the first latency, the processing circuitry can adjust the one or more parameters of the first parameter set to increase a subsequent latency for a subsequent ECAP signal elicited by a subsequent stimulation pulse. For example, the processing circuitry may increase the number of pulses in the next burst of pulses, increase stimulation amplitude of the subsequent pulses, or change the frequency of the pulses within the burst of pulses.

As described herein, the bursts of pulses may be conditioning pulses configured to condition nerves and suppress certain nerve fiber types from activation by subsequent stimulation pulses. For example, the first burst of pulses are configured to decrease an activation of a first set of nerve fibers of target tissue instead of a second set of nerve fibers of target tissues, and wherein the first parameter set defines the first burst of pulses comprising a sub-perception threshold intensity. For example, the conditioning pulses may decrease the activation of the first set of nerve fibers (e.g., fibers larger than the second set of fibers) by increasing the threshold of the first set of nerve fibers. The conditioning pulses may not affect, or minimally affect, the threshold of the second set of smaller nerve fibers. In some examples, the bursts of pulses for conditioning may have a pulse frequency selected in the range of 200 Hz to 1200 Hz, but higher or lower frequencies may be used in other examples. The bursts of pulses may be configured to have an intensity less than a perception threshold. This intensity may be defined by an amplitude and/or pulse width, and the perception threshold may be the intensity (or amplitude or pulse width) at which the pulses are perceptible by the patient. Pulses having this lower intensity may be referred to as sub-perception threshold or sub-threshold stimulation pulses. The system may adjust any parameters of the burst of pulses, such as the pulse amplitude (voltage amplitude or current amplitude), number of pulses in each burst, the frequency of the pulses within each burst, the pulse widths, or any other parameter that defines the pulses of each burst of pulses. In some examples, the amplitude of the burst of pulses may be lower than the amplitude of the stimulation pulse that follows. However, the amplitudes of both pulses may be based on a sweep of amplitudes and which amplitudes cause largest peaks in the ECAP signal indicative of the larger and smaller fibers to be activated.

The stimulation pulse that follows the burst of pulses may be delivered at a slower frequency, such as a pulse frequency from 10 Hz to 60 Hz. In this manner, the stimulation pulse (e.g., a control pulse) may be delivered in an interleaved pattern with one or more bursts of pulses such that stimulation pulses are delivered between bursts of pulses. However, there may be multiple bursts of pulses between consecutive stimulation pulses and/or multiple stimulation pulses delivered between consecutive bursts of pulses.

In some examples, the system may be configured to cycle the bursts of pulses on and off over time. Since the bursts of pulses may be used to condition target types of fibers, such as larger fibers that cause paresthesia, the system may refrain from, or withhold, delivery of one or more bursts of pulses when the conditioning effect is still occurring. This may be due to the conditioning effect lasting for seconds, minutes, or even hours in some examples. In one example, the processing circuitry may be configured to compare a latency indicated by the ECAP characteristic to a threshold latency and determine that the latency is greater than the threshold latency. Then, responsive to determining that the latency is greater than the threshold latency, the processing circuitry can withhold further bursts of pulses during a first period time in which additional stimulation pulses are delivered. In other words, the latency is greater than the threshold latency which renders additional bursts of pulse unnecessary because the larger fibers are already conditioned (e.g., the activation threshold has been raised). The processing circuity can then receive information representative of ECAP signals elicited by at least some of the additional stimulation pulses, determine ECAP characteristics of the ECAP signals that are indicative of respective latencies of the one or more nerve fibers activated by the at least some additional stimulation pulses, and responsive to determining that one of the respective latencies becomes shorter than the threshold latency, control delivery of additional bursts of pulses during a second period of time subsequent to the first period of time.

In some examples, system 100 can determine initial parameters for the bursts of pulses and/or the stimulation pulses based on a differential ECAP characteristic (e.g., a differential latency) indicative of the latency of a plurality of different fiber types activated by the different stimulation pulses. The processing circuitry can obtain the differential ECAP characteristic by receiving information representative of a plurality of ECAP signals elicited by different stimulation pulses defined by different parameter values. Based on these plurality of ECAP signals, the processing circuitry can determine the differential ECAP characteristic. Then, the processing circuitry can determine, based on the differential ECAP characteristic, at least one parameter value of the second parameter set defining the stimulation pulse. The processing circuitry can then determine, based on the differential ECAP characteristic, at least one parameter value of the first parameter set defining the first burst of pulses. The differential ECAP characteristic may be indicative of the difference between a peak occurring early in the ECAP signal and another peak occurring later in the ECAP signal, where the later peak is indicative of the longer latency in the smaller fibers.

FIG. 2 is a block diagram of IMD 200. IMD 200 may be an example of IMD 110 of FIG. 1. In the example shown in FIG. 2, IMD 200 includes stimulation generation circuitry 204, sensing circuitry 206, processing circuitry 208, sensor 210, telemetry circuitry 212, power source 214, and memory 216. Each of these circuits may be, or include, programmable or fixed function circuitry that can perform the functions attributed to respective circuitry. For example, processing circuitry 208 may include fixed-function or programmable circuitry, stimulation generation circuitry 204 may include circuitry that can generate electrical stimulation signals such as pulses or continuous waveforms on one or more channels, sensing circuitry 206 may include sensing circuitry for sensing signals, and telemetry circuitry 212 may include telemetry circuitry for transmission and reception of signals. Memory 216 may store computer-readable instructions that, when executed by processing circuitry 208, cause IMD 200 to perform various functions described herein. Memory 216 may be a storage device or other non-transitory medium.

In the example shown in FIG. 2, memory 216 stores patient data 218, which may include parameters associated with the patient such as one or more patient postures, an activity level, or a combination of patient posture and activity level. A set of pre-established posture state definitions for a patient may be stored in patient data 218. A posture state definition may be modified based on user therapy adjustments and/or posture state information. In some cases, the posture state may be expanded and split, or instead, may be reduced in size based on posture state information. The posture state definitions can be automatically updated or updated by a patient, including creating new posture states. Posture states may include, for example, a supine posture, a prone posture, a lying left and/or lying right, a sitting posture, a reclining posture, a standing posture, and/or activities such as running or riding in an automobile.

Memory 216 may store stimulation parameter settings 220 within memory 216 or separate areas within memory 216. Each stored stimulation parameter setting 220 defines values for a set of electrical stimulation parameters (e.g., a stimulation parameter set or therapy program), such as pulse amplitude, pulse width, pulse frequency, electrode combination, pulse burst rate, pulse burst duration, and/or waveform shape for any stimulation and type of pulses, such as the bursts of pulses and stimulation pulses that follow. Stimulation parameter settings 220 may also include additional information such as instructions regarding delivery of electrical stimulation signals based on stimulation parameter relationship data, which can include relationships between two or more stimulation parameters based upon data from electrical stimulation signals delivered to patient 102 or data transmitted from external programmer 104. The stimulation parameter relationship data may include measurable aspects associated with stimulation, such as an ECAP characteristic value.

Memory 216 also stores closed-loop instructions 222 which may include instructions for IMD 200 regarding how to adjust stimulation parameters based on sensed data, such as ECAP signals. For example, closed-loop instructions 222 may also include target ECAP characteristics and/or threshold ECAP characteristic values determined for the patient and/or a history of measured ECAP characteristic values for the patient. The closed-loop instructions 222 may include latency thresholds, differential ECAP characteristics (e.g., differential latencies), or any other values required for processing circuitry 208 to operate in a closed-loop manner as described herein. Memory 216 may also store latency data 224 in separate areas from or as part of patient stimulation parameter settings. Latency data 224 may include raw data of the latencies for ECAP signals, differential latencies, or any other ECAP characteristics associated with ECAP signals obtained by IMD 200.

Accordingly, in some examples, stimulation generation circuitry 204 generates electrical stimulation signals (e.g., pulses) in accordance with the electrical stimulation parameters noted above. Other ranges of stimulation parameter values may also be useful and may depend on the target stimulation site within patient 102. While stimulation pulses are described, stimulation signals may be of any form, such as continuous-time signals (e.g., sine waves) or the like. Stimulation generation circuitry 204 may include independently controllable current sinks and sources for respective electrodes 232, 234. For example, stimulation generation circuitry 204 comprises a plurality of pairs of voltage sources, current sources, voltage sinks, or current sinks connected to each of electrodes 232, 234 such that each pair of electrodes has a unique signal circuit. In other words, in these examples, each of electrodes 232, 234 is independently controlled via its own signal circuit (e.g., via a combination of a regulated voltage source and sink or regulated current source and sink), as opposed to switching signals between electrodes 232, 234. In this manner, processing circuitry 208 may control switches or transistors to selective couple the sources and/or sinks to the conductor of electrodes of an electrode combination.

One or more switches (not shown) may selectively couple sensing circuitry 206 to respective electrodes in order to sense signals via two or more electrodes 232, 234. In other examples, switch circuitry may include one or more switch arrays, one or more multiplexers, one or more switches (e.g., a switch matrix or other collection of switches), or other electrical circuitry configured to direct stimulation signals from stimulation generation circuitry 204 to one or more of electrodes 232, 234, or directed sensed signals from one or more of electrodes 232, 234 to sensing circuitry 206. In other examples, stimulation generation circuitry 204 and/or sensing circuitry 206 may include sensing circuitry to direct signals to and/or from one or more of electrodes 232, 234, which may or may not also include switch circuitry.

Sensing circuitry 206 may be configured to monitor signals from any combination of electrodes 232, 234. In some examples, sensing circuitry 206 includes one or more amplifiers, filters, and analog-to-digital converters. Sensing circuitry 206 may be used to sense physiological signals, such as ECAPs. In some examples, sensing circuitry 206 detects ECAPs from a particular combination of electrodes 232, 234. In some cases, the particular combination of electrodes for sensing ECAPs includes different electrodes than a set of electrodes 232, 234 used to deliver stimulation pulses. Alternatively, in other cases, the particular combination of electrodes used for sensing ECAPs includes at least one of the same electrodes as a set of electrodes used to deliver stimulation pulses to patient 102. Sensing circuitry 206 may provide signals to an analog-to-digital converter, for conversion into a digital signal for processing, analysis, storage, or output by processing circuitry 208.

Processing circuitry 208 may include any one or more of a microprocessor, a controller, a digital signal processor (DSP), an application specific integrated circuit (ASIC), a field-programmable gate array (FPGA), discrete logic circuitry, or any other processing circuitry can provide the functions attributed to processing circuitry 208 herein may be embodied as firmware, hardware, software, or any combination thereof. Processing circuitry 208 controls stimulation generation circuitry 204 to generate electrical stimulation signals according to stimulation parameter settings 220 stored in memory 216 to apply stimulation parameter values, such as pulse amplitude, pulse width, pulse frequency, and waveform shape of each of the electrical stimulation signals.

In the example shown in FIG. 2, the set of electrodes 232 includes electrodes 232A, 232B, 232C, and 232D, and the set of electrodes 234 includes electrodes 234A, 234B, 234C, and 234D. In other examples, a single lead may include all eight electrodes 232 and 234 along a single axial length of the lead. Processing circuitry 208 also controls stimulation generation circuitry 204 to generate and apply the electrical stimulation signals to selected combinations of electrodes 232, 234. In other examples, stimulation generation circuitry 204 includes a switch circuit that may couple stimulation signals to selected conductors within leads 230, which, in turn, deliver the stimulation signals across selected electrodes 232, 234. Such a switch circuit may be a switch array, switch matrix, multiplexer, or any other type of switch circuitry can selectively couple stimulation energy to selected electrodes 232, 234 and to selectively sense bioelectrical neural signals of a spinal cord of the patient (not shown in FIG. 2) with selected electrodes 232, 234.

Electrodes 232, 234 on respective leads 230 may be constructed of a variety of different designs. For example, one or both of leads 230 may include one or more electrodes at each longitudinal location along the length of the lead, such as one electrode at different perimeter locations around the perimeter of the lead at each of the locations A, B, C, and D. In one example, the electrodes may be electrically coupled to stimulation generation circuitry 204, e.g., via switch circuitry 202 and/or switch circuitry of the stimulation generation circuitry 204, via respective wires that are straight or coiled within the housing of the lead and run to a connector at the proximal end of the lead. In another example, each of the electrodes of the lead may be electrodes deposited on a thin film. The thin film may include an electrically conductive trace for each electrode that runs the length of the thin film to a proximal end connector. The thin film may then be wrapped (e.g., a helical wrap) around an internal member to form the lead 230. These and other constructions may be used to create a lead with a complex electrode geometry. In other examples, optical fiber or optical transfers may be used to sense ECAP signals as described herein.

Although sensing circuitry 206 is incorporated into a common housing with stimulation generation circuitry 204 and processing circuitry 208 in FIG. 2, in other examples, sensing circuitry 206 may be in a separate housing from IMD 200 and may communicate with processing circuitry 208 via wired or wireless communication techniques. In some examples, one or more of electrodes 232 and 234 may be suitable for sensing ECAPs. For instance, electrodes 232 and 234 may sense the voltage amplitude of a portion of the ECAP signals, where the sensed voltage amplitude is a characteristic the ECAP signal.

Memory 216 may be configured to store information within IMD 200 during operation. Memory 216 may include a computer-readable storage medium or computer-readable storage device. In some examples, memory 216 includes one or more of a short-term memory or a long-term memory. Memory 216 may include, for example, random access memories (RAM), dynamic random access memories (DRAM), static random access memories (SRAM), magnetic discs, optical discs, flash memories, or forms of electrically programmable memories (EPROM) or electrically erasable and programmable memories (EEPROM). In some examples, memory 216 is used to store data indicative of instructions for execution by processing circuitry 208. As discussed herein, memory 216 can store patient posture state data 218, stimulation parameter settings 220, calibration instructions 222, and latency data 224.

Sensor 210 may include one or more sensing elements that sense values of a respective patient parameter. As described, electrodes 232 and 234 may be the electrodes that sense, via sensing circuitry 206, a value of the ECAP indicative of a target stimulation intensity at least partially caused by a set of stimulation parameter values. Sensor 210 may include one or more accelerometers, optical sensors, chemical sensors, temperature sensors, pressure sensors, or any other types of sensors. Sensor 210 may output patient parameter values that may be used as feedback to control delivery of electrical stimulation signals. IMD 200 may include additional sensors within the housing of IMD 200 and/or coupled via one of leads 108 or other leads. In addition, IMD 200 may receive sensor signals wirelessly from remote sensors via telemetry circuitry 212, for example. In some examples, one or more of these remote sensors may be external to patient (e.g., carried on the external surface of the skin, attached to clothing, or otherwise positioned external to the patient). In some examples, signals from sensor 210 may indicate a posture state (e.g., sleeping, awake, sitting, standing, or the like), and processing circuitry 208 may select target and/or threshold ECAP characteristic values according to the indicated posture state. In this manner, processing circuitry 208 may be configured to determine the currently occupied posture state of patient 102.

Telemetry circuitry 212 supports wireless communication between IMD 200 and an external programmer (not shown in FIG. 2) or another computing device under the control of processing circuitry 208. Processing circuitry 208 of IMD 200 may receive, as updates to programs, values for various stimulation parameters such as amplitude and electrode combination, from the external programmer via telemetry circuitry 212. Updates to stimulation parameter settings 220 and input efficacy threshold settings 226 may be stored within memory 216. Telemetry circuitry 212 in IMD 200, as well as telemetry circuits in other devices and systems described herein, such as the external programmer, may accomplish communication by radiofrequency (RF) communication techniques. In addition, telemetry circuitry 212 may communicate with an external medical device programmer (not shown in FIG. 2) via proximal inductive interaction of IMD 200 with the external programmer. The external programmer may be one example of external programmer 104 of FIG. 1. Accordingly, telemetry circuitry 212 may send information to the external programmer on a continuous basis, at periodic intervals, or upon request from IMD 110 or the external programmer.

Power source 214 delivers operating power to various components of IMD 200. Power source 214 may include a rechargeable or non-rechargeable battery and a power generation circuit to produce the operating power. Recharging may be accomplished through proximal inductive interaction between an external charger and an inductive charging coil within IMD 200. In other examples, traditional primary cell batteries may be used. In some examples, processing circuitry 208 may monitor the remaining charge (e.g., voltage) of power source 214 and select stimulation parameter values that may deliver similarly effective therapy at lower power consumption levels when needed to extend the operating time of power source 214. For example, power source 214 may switch to a lower pulse frequency based on the relationships of parameters that may provide similar ECAP characteristic values.

According to the techniques of the disclosure, stimulation generation circuitry 204 of IMD 200 receives, via telemetry circuitry 212, instructions to deliver electrical stimulation according to stimulation parameter settings 220 to a target tissue site of the spinal cord of the patient via a plurality of electrode combinations of electrodes 232, 234 of leads 230 and/or a housing of IMD 200. Each electrical stimulation signal may elicit an ECAP that is sensed by sensing circuitry 206 via electrodes 232 and 234. Processing circuitry 208 may receive, via an electrical signal sensed by sensing circuitry 206, information indicative of an ECAP signal (e.g., a numerical value indicating a characteristic of the ECAP in electrical units such as voltage or power) produced in response to the electrical stimulation signal(s). Stimulation parameter settings 220 may be updated according to the ECAPs recorded at sensing circuitry 206 according to the following techniques.

In one example, the bursts of pulses each have a pulse width in a range of 50 μs to 500 μs. In some examples, the pulses within each burst may have a pulse with of approximately 400 μs. The inter-burst period or burst frequency may be selected such that a stimulation can be delivered, and elicited ECAP sensed, between consecutive bursts of pulses. In one example, each stimulation pulse (e.g., a control pulse) following the bursts of pulses may have a pulse width in a range of 50 μs to 250 μs. In some examples, the stimulation pulses may be approximately 200 μs. The stimulation pulses may have a pulse frequency in a range of approximately 10 Hz to 60 Hz. Amplitude (current and/or voltage) for the pulses may be between approximately 0.5 mA (or volts) and approximately 10 mA (or volts), although amplitude may be lower or greater in other examples.

Processing circuitry 208 may be configured to compare one or more characteristics of ECAPs sensed by sensing circuitry 206 with target ECAP characteristics stored in memory 216 (e.g., patient ECAP characteristics 222). For example, processing circuitry 208 can determine the amplitude of each ECAP signal received at sensing circuitry 206, and processing circuitry 208 can determine the representative amplitude of at least one respective ECAP signal and compare the representative amplitude of a series of ECAP signals to a target ECAP. In some examples, processing circuitry 208 may compare the latencies of respective ECAP characteristics or a latency characteristic of multiple ECAP signals to prior latencies or a threshold latency.

In other examples, processing circuitry 208 may use the representative amplitude of the at least one respective ECAP to change other parameters of stimulation pulses to be delivered, such as pulse width, pulse frequency, and pulse shape. All of these parameters may contribute to the intensity of the stimulation pulses, and changing one or more of these parameter values may effectively adjust the stimulation pulse intensity to compensate for the changed distance between the stimulation electrodes and the nerves indicated by the characteristic (e.g., a representative amplitude) of the ECAP signals.

In some examples, leads 230 may be linear 8-electrode leads (not pictured); sensing and stimulation delivery may each be performed using a different set of electrodes. In a linear 8-electrode lead, each electrode may be numbered consecutively from 0 through 7. For instance, a pulse may be generated using electrode 1 as a cathode and electrodes 0 and 2 as anodes (e.g., a guarded cathode), and a respective ECAP signal may be sensed using electrodes 6 and 7, which are located on the opposite end of the electrode array. This strategy may minimize the interference of the stimulation pulse with the sensing of the respective ECAP. Other electrode combinations may be implemented, and the electrode combinations may be changed using the patient programmer via telemetry circuitry 212. For example, stimulation electrodes and sensing electrodes may be positioned closer together. Shorter pulse widths for the nontherapeutic pulses may allow the sensing electrodes to be closer to the stimulation electrodes.

In one example, sensor 210 may detect a change in posture state, including activity or a change in posture of the patient. Processing circuitry 208 may receive an indication from sensor 210 that the activity level or posture of the patient is changed, and processing circuitry 208 can initiate or change the delivery of the plurality of pulses according to stimulation parameter settings 220. For example, processing circuitry 208 may increase the frequency of pulse delivery and respective ECAP sensing in response to receiving an indication that the patient activity has increased, which may indicate that the distance between electrodes and nerves will likely change. Alternatively, processing circuitry 208 may decrease the frequency of pulse delivery and respective ECAP sensing in response to receiving an indication that the patient activity has decreased. In some examples, one or more therapy parameters (e.g., frequency, amplitude, slew rate, pulse width, or the like) may be adjusted (e.g., increased or decreased) in response to receiving an indication that the patient posture state has changed.

Processing circuitry 208 can update patient posture state data 218 and latency data 224 according to the signal received from sensor 210.

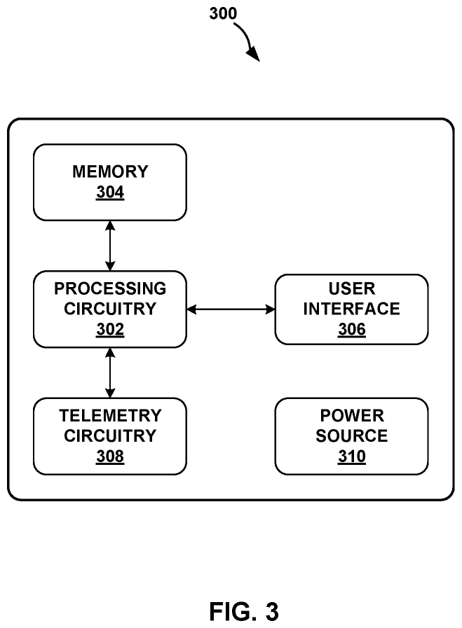

FIG. 3 is a block diagram of the example external programmer 300. External programmer 300 may be an example of external programmer 104 of FIG. 1. Although programmer 300 may generally be described as a hand-held device, external programmer 300 may be a larger portable device or a more stationary device. In addition, in some examples, external programmer 300 may be included as part of an external charging device or include the functionality of an external charging device. As illustrated in FIG. 3, external programmer 300 may include a processing circuitry 302, memory 304, user interface 306, telemetry circuitry 308, and power source 310. Storage device 304 may store instructions that, when executed by processing circuitry 302, cause processing circuitry 302 and external programmer 300 to provide the functionality ascribed to external programmer 300 throughout this disclosure. Each of these components, circuitry, or modules, may include electrical circuitry that can perform some, or all of the functionality described herein. For example, processing circuitry 302 may include processing circuitry to perform the processes discussed with respect to processing circuitry 302.

In general, programmer 300 comprises any suitable arrangement of hardware, alone or in combination with software and/or firmware, to perform the techniques attributed to programmer 300, and processing circuitry 302, user interface 306, and telemetry circuitry 308 of programmer 300. In various examples, programmer 300 may include one or more processors, such as one or more microprocessors, DSPs, ASICs, FPGAs, or any other equivalent integrated or discrete logic circuitry, as well as any combinations of such components. Programmer 300 also, in various examples, may include a memory 304, such as RAM, ROM, PROM, EPROM, EEPROM, flash memory, a hard disk, a CD-ROM, comprising executable instructions for causing the one or more processors to perform the actions attributed to them. Moreover, although processing circuitry 302 and telemetry circuitry 308 are described as separate, in some examples, processing circuitry 302 and telemetry circuitry 308 are functionally integrated. In some examples, processing circuitry 302 and telemetry circuitry 308 correspond to individual hardware units, such as ASICs, DSPs, FPGAs, or other hardware units.

Memory 304 (e.g., a storage device) may store instructions that, when executed by processing circuitry 302, cause processing circuitry 302 and programmer 300 to provide the functionality ascribed to programmer 300 throughout this disclosure. For example, memory 304 may include instructions that cause processing circuitry 302 to obtain a stimulation parameter setting from memory, select a spatial electrode movement pattern, or receive a user input and send a corresponding command to programmer 300, or instructions for any other functionality. In addition, memory 304 may include a plurality of stimulation parameter settings, where each setting includes a parameter set that defines electrical stimulation. Memory 304 may also store data received from a medical device (e.g., IMD 110). For example, memory 304 may store ECAP related data recorded at a sensing circuitry of the medical device, and memory 304 may also store data from one or more sensors of the medical device.

User interface 306 may include a button or keypad, lights, a speaker for voice commands, a display, such as a liquid crystal (LCD), light-emitting diode (LED), or organic light-emitting diode (OLED). In some examples the display may be a touch screen. User interface 306 can display any information related to the delivery of electrical stimulation, identified patient behaviors, sensed patient parameter values, patient behavior criteria, or any other such information. External programmer 300 may receive user input (e.g., indication of when the patient changes posture states) via user interface 306. The input may be, for example, in the form of pressing a button on a keypad or selecting an icon from a touch screen. The input may request starting or stopping electrical stimulation, the input may request a new spatial electrode movement pattern or a change to an existing spatial electrode movement pattern, of the input may request some other change to the delivery of electrical stimulation. In other examples, user interface 306 may receive input from the patient and/or clinician regarding efficacy of the therapy, such as binary feedback, numerical ratings, textual input, etc. In some examples, processing circuitry 302 may interpret patient requests to change therapy as negative feedback regarding the current parameter values used to define therapy.

Telemetry circuitry 308 may support wireless communication between the medical device and programmer 300 under the control of processing circuitry 302. Telemetry circuitry 308 can communicate with another computing device via wireless communication techniques, or direct communication through a wired connection. In some examples, telemetry circuitry 308 provides wireless communication via an RF or proximal inductive medium. In some examples, telemetry circuitry 308 includes an antenna, which may take on a variety of forms, such as an internal or external antenna.

Examples of local wireless communication techniques that may be employed to facilitate communication between programmer 300 and IMD 110 include RF communication according to the 802.11 or Bluetooth specification sets or other standard or proprietary telemetry protocols. In this manner, other external devices may be capable of communicating with programmer 300 without needing to establish a secure wireless connection. As described herein, telemetry circuitry 308 can transmit a spatial electrode movement pattern or other stimulation parameter values to IMD 110 for delivery of electrical stimulation.

In some examples, selection of stimulation parameter settings may be transmitted to the medical device for delivery to the patient. In other examples, stimulation parameter settings may include medication, activities, or other instructions that the patient must perform themselves or a caregiver perform for patient 102. In some examples, external programmer 300 may provide visual, audible, and/or tactile notifications that indicate there are new instructions.

External programmer 300 may require receiving user input acknowledging that the instructions have been completed in some examples.

According to the techniques of the disclosure, user interface 306 of external programmer 300 receives an indication from a clinician instructing a processor of the medical device to update one or more stored values, such as requesting recalibration of the relationships between stimulation parameter values and ECAP signal feature latencies, patient posture state settings, gain values, growth curve settings, or stimulation parameter settings. User interface 306 may also receive instructions from the clinician commanding any electrical stimulation. For example, user interface 306 may receive an indication that therapy is no longer effective or side effects have occurred such as feeling paresthesia where the therapy is attempting to minimize paresthesia. Processing circuitry 208 may directly determine, or control IMD 200 to determine, adjusted parameter values for the bursts of pulses and/or the stimulation pulse in order to regain therapy efficacy.

Power source 310 can deliver operating power to various components of programmer 300. Power source 310 may be the same as or substantially similar to power source 214. Power source 310 may include a battery and a power generation circuit to produce the operating power. In some examples, the battery is rechargeable to allow extended operation. Recharging may be accomplished by electrically coupling power source 310 to a cradle or plug that is connected to an alternating current (AC) outlet. In addition, recharging may be accomplished through proximal inductive interaction between an external charger and an inductive charging coil within external programmer 300. In other examples, traditional batteries (e.g., nickel cadmium or lithium ion batteries) may be used. In addition, external programmer 300 may be directly coupled to an alternating current outlet to operate.

The architecture of external programmer 300 illustrated in FIG. 3 is shown as an example. The techniques as set forth in this disclosure may be implemented in the example external programmer 300 of FIG. 3, as well as other types of systems not described specifically herein. Nothing in this disclosure should be construed so as to limit the techniques of this disclosure to the example architecture illustrated by FIG. 3.

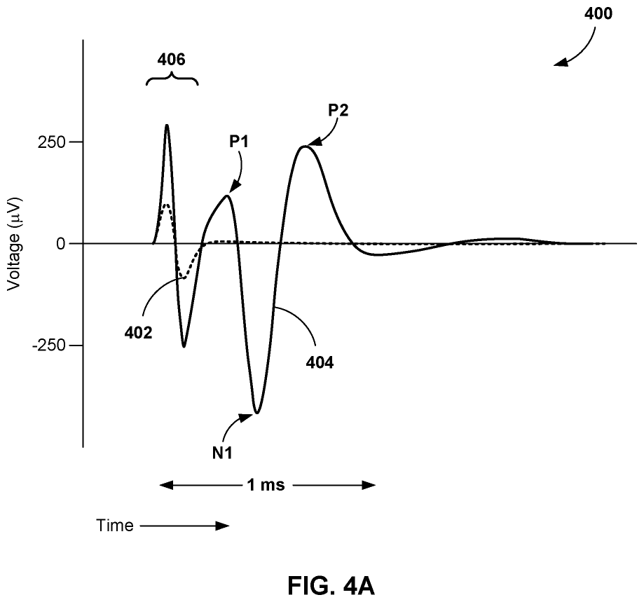

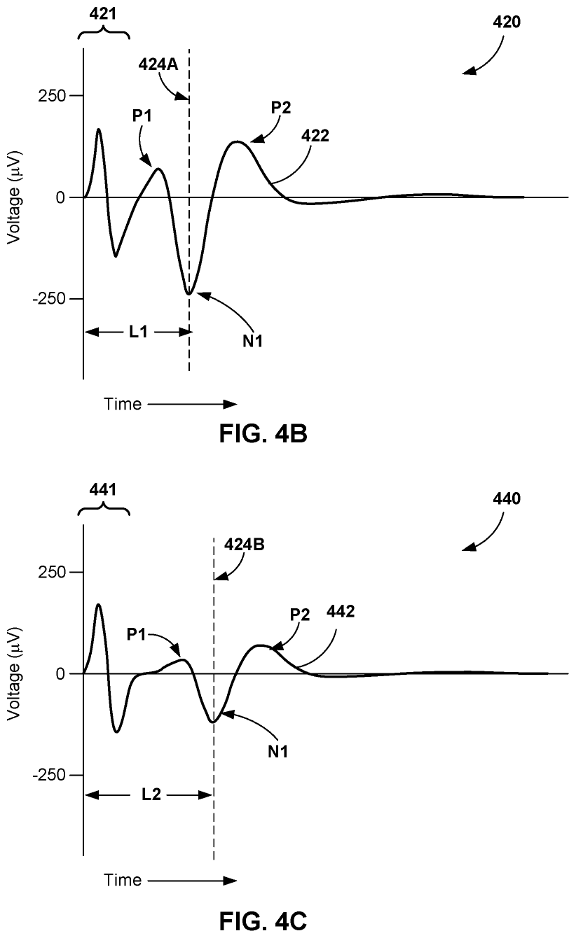

FIG. 4A is a graph 400 of an example ECAP signals sensed for respective electrical stimulation pulses. As shown in FIG. 4A, graph 400 shows example ECAP signal 402 (dotted line) and ECAP signal 404 (solid line). Each of ECAP signals 402 and 404 may be sensed from pulses that were delivered from a guarded cathode and bi-phasic pulses including an interphase interval between each positive and negative phase of the pulse. The guarded cathode of the stimulation electrodes is located at the end of an 8-electrode lead while two sensing electrodes are provided at the other end of the 8-electrode lead. ECAP signal 402 illustrates the voltage amplitude sensed as a result from a sub-threshold stimulation pulse. Peaks 406 of ECAP signal 402 are detected and represent the artifact of the delivered pulse. However, no propagating signal is detected after the artifact in ECAP signal 404 because the pulse was sub-threshold.

In contrast to ECAP signal 402, ECAP signal 404 represents the voltage amplitude detected from a supra-threshold stimulation pulse. Peaks 406 of ECAP signal 404 are detected and represent the artifact of the delivered pulse. After peaks 406, ECAP signal 404 also includes various features, which include peaks P1, N1, and P2, which are three peaks representative of propagating action potentials from an ECAP. In some examples, N1 may be referred to as a negative peak or trough instead. The example duration of the artifact and peaks P1, N1, and P2 is approximately 1 millisecond (ms). When detecting the ECAP of ECAP signal 404, different characteristics may be identified. For example, the characteristic of the ECAP may be the amplitude between features N1 and P2. This N1-P2 amplitude can be detected even if the artifact impinges on P1, a relatively large signal, and the N1-P2 amplitude may be minimally affected by electronic drift in the signal. In other examples, the characteristic of the ECAP used to control pulses may be an amplitude of P1, N1, or P2 with respect to neutral or zero voltage. In some examples, the characteristic of the ECAP used to control pulses may be a sum of two or more of peaks P1, N1, or P2. In other examples, the characteristic of ECAP signal 404 may be the area under one or more of peaks P1, N1, and/or P2. In other examples, the characteristic of the ECAP may be a ratio of one of peaks P1, N1, or P2 to another one of the peaks. In some examples, the characteristic of the ECAP may be a slope between two points in the ECAP signal, such as the slope between N1 and P2. In other examples, the characteristic of the ECAP may be the time between two points of the ECAP, such as the time between N1 and P2. The time between two points in the ECAP signal (e.g., the beginning of stimulation pulse of peaks 406 and N1) may be referred to as a latency of the ECAP and may indicate the types of fibers being captured by the pulse. ECAP signals with lower latency (i.e., smaller latency values) indicate a higher percentage of nerve fibers that have faster propagation of signals, whereas ECAP signals with higher latency (i.e., larger latency values) indicate a higher percentage of nerve fibers that have slower propagation of signals. Other characteristics of the ECAP signal may be used in other examples.

The amplitude of the ECAP signal increases with increased amplitude of the pulse, as long as the pulse amplitude is greater than the threshold such that nerves depolarize and propagate the signal. The target ECAP characteristic (e.g., the target ECAP amplitude) may be determined from the ECAP signal detected from a pulse when pulses are determined to deliver effective therapy to the patient. The ECAP signal thus is representative of the distance between the stimulation electrodes and the nerves appropriate for the stimulation parameter values of the pulses delivered at that time. Therefore, IMD 110 may attempt to use detected changes to the measured ECAP characteristic value to change stimulation pulse parameter values and maintain the target ECAP characteristic value during stimulation pulse delivery. Alternatively, IMD 110 may attempt to prevent undesirable stimulation intensity by decreasing stimulation pulse intensity in response to the ECAP characteristic value exceeding a threshold ECAP characteristic value.

FIGS. 4B and 4C are graphs of example ECAP signals sensed from stimulation pulses having different current amplitudes for different nerve fibers. Graph 420 of FIG. 4B is similar to graph 440 of FIG. 4C, but graph 440 indicates how latency L2 of ECAP signal 442 increases when compared to latency L1 of ECAP signal 422 for smaller nerve fibers. As shown in FIG. 4B, ECAP signal 422 includes stimulation pulse 421 and the following features of the ECAP signal which include P1, N1, and P2. Time 424A indicates the time at which N1 appears for the larger fibers, which defines latency L1 for the presence of N1.

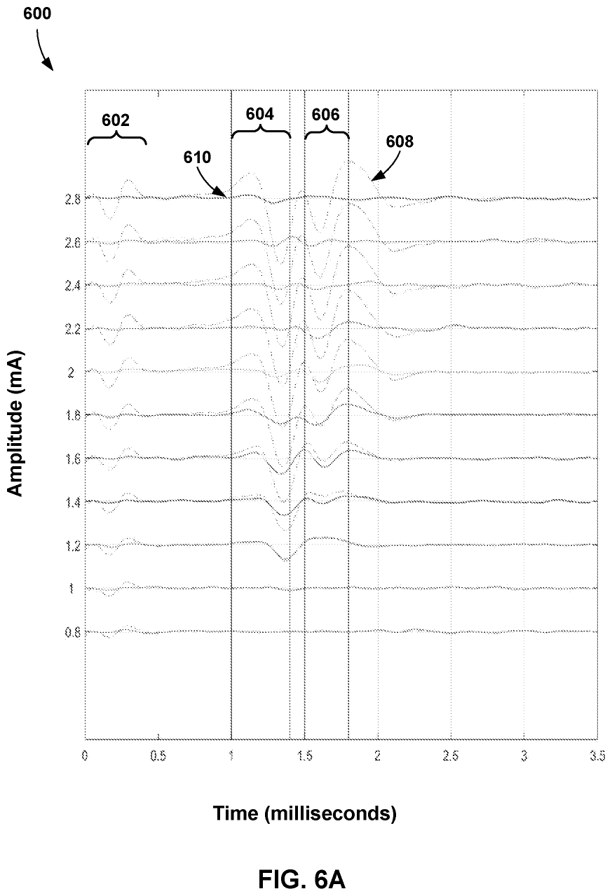

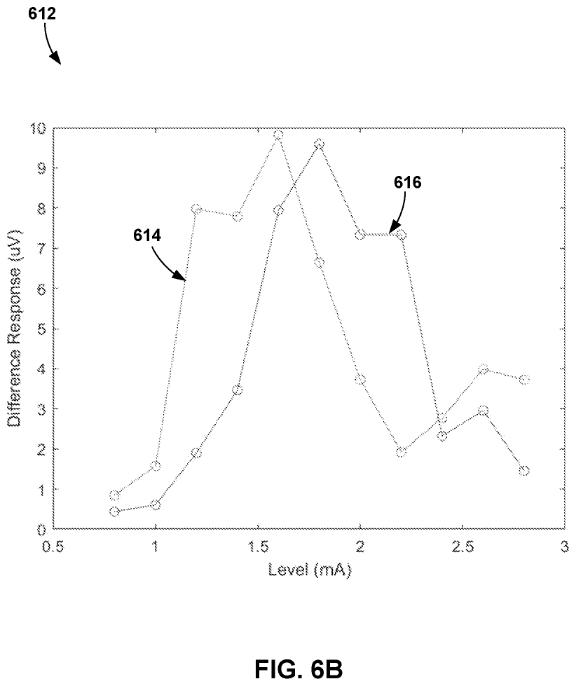

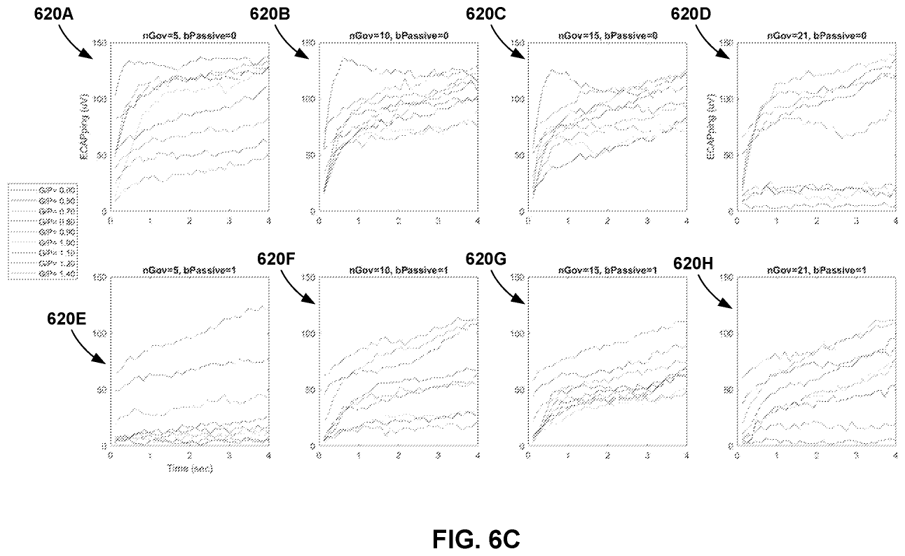

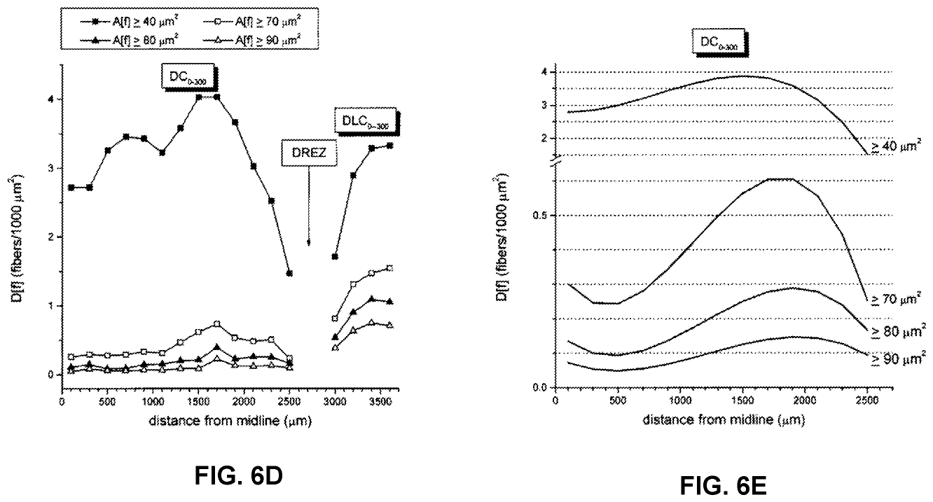

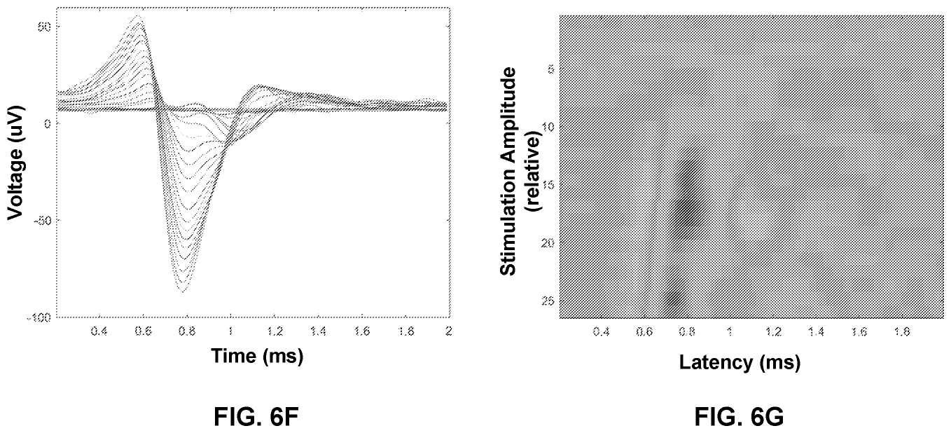

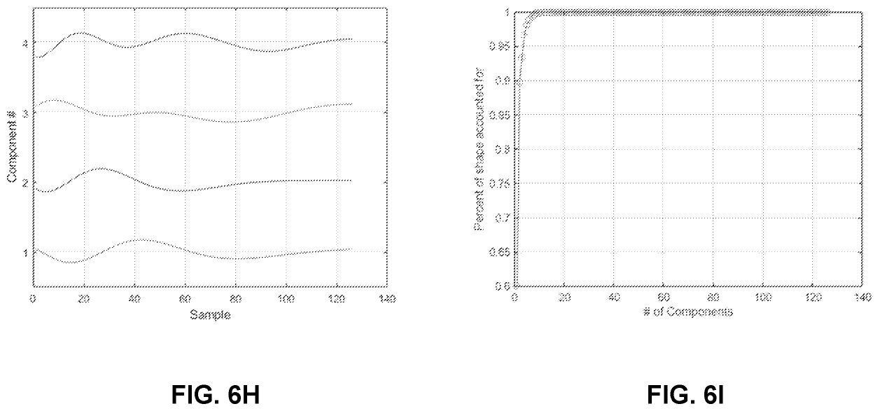

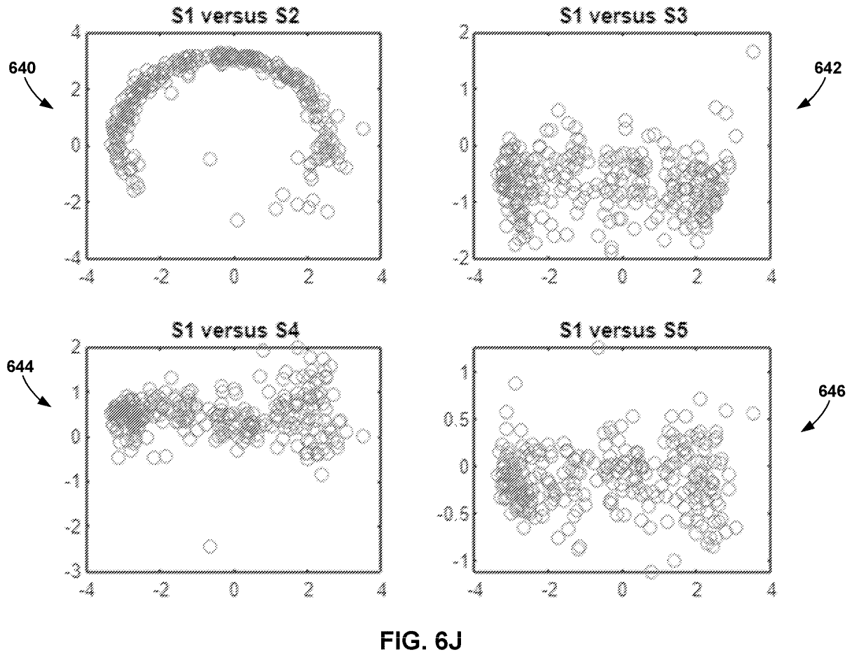

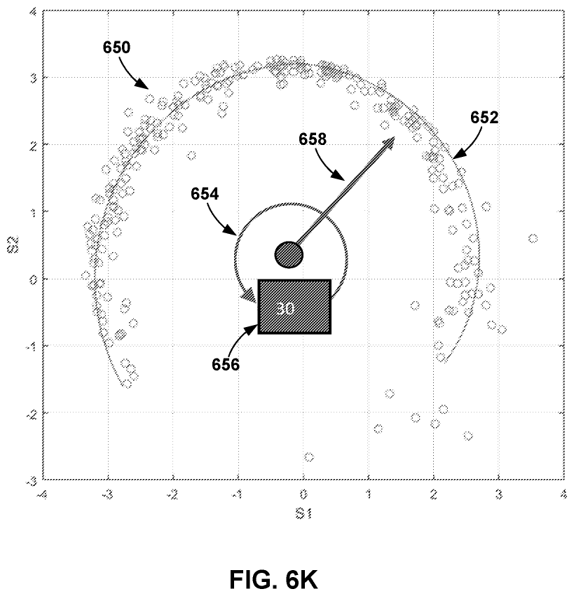

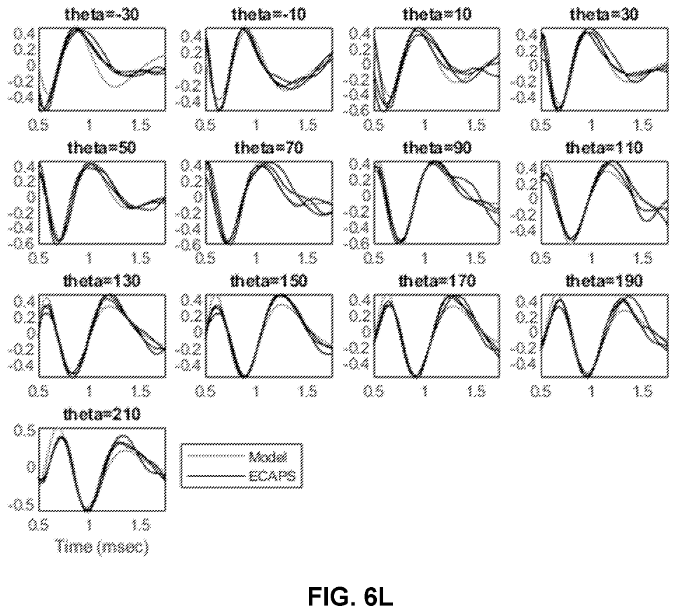

However, graph 440 of FIG. 4C illustrates an example ECAP signal 442 for smaller, slower nerve fibers than those responsible for ECAP signal 422 in FIG. 4B. Stimulation pulse 441 is shown to have a similar current amplitude than stimulation pulse 421. However, because the response from smaller fibers is shown, latency L2 to time 424B is longer, or has increased, compared to L1 in FIG. 4B for larger nerve fibers. Therefore, the system may utilize this difference in latencies of the peaks in ECAP signals for different nerve fibers to identify different latencies and different rations of nerve fibers activated by the stimulation pulse delivered. ECAP signals 422 and 442 are idealized and separated for each types of fibers. In reality, the system would sense these signals 422 and 442 super imposed on each other because the sensed ECAP will be representative of all fibers activated by the stimulation pulses. FIG. 6A illustrates example ECAP signals indicative of different ratios of types of fibers.

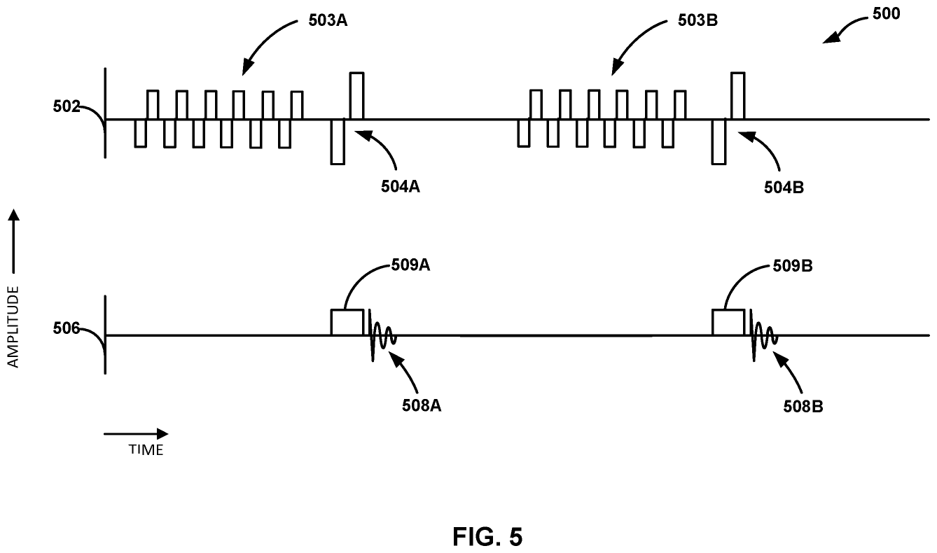

FIG. 5 is a timing diagram 500 illustrating one example of electrical stimulation pulses and respective sensed ECAPs, in accordance with one or more techniques of this disclosure. For convenience, FIG. 5 is described with reference to IMD 200 of FIG. 2. As illustrated, timing diagram 500 includes first channel 502, a plurality of control pulses 504A-504B (collectively “control pulses 504”) and a plurality of bursts of pulses 503A and 503B (collectively “bursts 503), second channel 506, a plurality of respective ECAPs 508A-508B (collectively “ECAPs 508”), and a plurality of stimulation interference signals 509A-509B (collectively “stimulation interference signals 509”). In the example of FIG. 5, bursts 503 are bursts of pulses configured to condition certain types of nerve fibers (e.g., larger fibers), such as increase the activation threshold of these types of nerve fibers so that stimulation pulses 504 are less likely to activate those certain types of fibers. Although each of bursts 503 include six pulses, bursts 503 may include fewer or more pulses within each burst. Stimulation pulses 504 may then activate other nerve fibers, such as smaller fibers associated with anesthesia, instead of the conditioned larger fibers of the certain types of fibers. Stimulation pulses 504 may also elicit respective ECAPs 508 for the purpose of determining relative neural recruitment due to the stimulation pulses 504 and latencies of the signal, which may be reflective of which nerve fibers, or a relative ratio of fibers, actually activated by the respective stimulation pulse 504.

First channel 502 is a time/voltage (and/or current) graph indicating the voltage (or current) of at least one electrode of electrodes 232, 234. In one example, the stimulation electrodes of first channel 502 may be located on the opposite side of the lead as the sensing electrodes of second channel 506. Stimulation pulses 504 may be electrical pulses delivered to the spinal cord of the patient by at least one of electrodes 232, 234, and stimulation pulses 504 may be balanced biphasic square pulses with an interphase interval. In other words, each of control pulses 504 are shown with a negative phase and a positive phase separated by an interphase interval. For example, a control pulse 504 may have a negative voltage for the same amount of time and amplitude that it has a positive voltage. It is noted that the negative voltage phase may be before or after the positive voltage phase. Stimulation pulses 504 may be delivered according to instructions stored in storage device 212 of IMD 200. Bursts 503 and stimulation pulses 504 may be delivered by the same electrode combinations. In other examples, bursts 503 may be delivered by a certain electrode combination of one or more anodes and one or more cathodes, and stimulation pulses 504 may be delivered by a different electrode combination of one or more anodes and one or more cathodes.

In some examples, each of stimulation pulses 504 may be a part of a sweep of pulses configured to determine latencies caused by different stimulation parameter values of the stimulation pulses 504 and/or different parameter values for different bursts 503. In this manner, each of stimulation pulses 504 may differ from each other by a parameter value, such as an iteratively increasing current amplitude. In some examples, the sweep may also include iteratively decreasing current amplitude, or a separate sweep of iteratively decreasing current amplitude may be performed. Separate latency curves (e.g., differential latencies or differential ECAP signals) may be generated from the respective increasing and decreasing current amplitudes in order to adjust the sensing window based on whether or not the stimulation parameter value is increasing or decreasing from the previous stimulation pulse. In some examples, such sweeps may be performed for each posture state of a plurality of posture states in order to determine the latency curves or some characteristic related to ECAPs for that posture state. As illustrated in FIG. 5, stimulation pulses 504 may be delivered via channel 502.

Delivery of stimulation pulses 504 may be delivered by leads 230 in a guarded cathode electrode combination. For example, if leads 230 are linear 8-electrode leads, a guarded cathode combination is a central cathodic electrode with anodic electrodes immediately adjacent to the cathodic electrode. The pulse frequency of the pulses within each of bursts 503 may be in a range of 200 Hz to 1200 Hz, or lower or higher in other examples. The pulse frequency of stimulation pulses 504 may be in a range of 40 Hz to 60 Hz in some examples. In some examples, a burst 503 may not be delivered between consecutive stimulation pulses 504 if a previous burst 503 caused conditioning that lasts longer than the period between consecutive stimulation pulses 504.

Second channel 506 is a time/voltage (and/or current) graph indicating the voltage (or current) of at least one electrode of electrodes 232, 234. In one example, the electrodes of second channel 506 may be located on the opposite side of the lead as the electrodes of first channel 502. ECAPs 508 may be sensed at electrodes 232, 234 from the spinal cord of the patient in response to stimulation pulses 504. ECAPs 508 are electrical signals which may propagate along a nerve away from the origination of stimulation pulses 504. In one example, ECAPs 508 are sensed by different electrodes than the electrodes used to deliver stimulation pulses 504. As illustrated in FIG. 5, ECAPs 508 may be recorded on second channel 506. In some examples, ECAPs 508 may not be sensed after each stimulation pulse 504.

Stimulation interference signals 509A and 509B (e.g., the artifact of the stimulation pulses) may be sensed by leads 230 and may be sensed during the same period of time as the delivery of stimulation pulses 504. Since the interference signals may have a greater amplitude and intensity than ECAPs 508, any ECAPs arriving at IMD 200 during the occurrence of stimulation interference signals 509 may not be adequately sensed by sensing circuitry 206 of IMD 200. However, ECAPs 508 may be sufficiently sensed by sensing circuitry 206 because each ECAP 508, or at least a portion of ECAP 508 that includes one or more desired features of ECAP 508 that is used to detect the posture state and/or as feedback for stimulation pulses 504, falls after the completion of each a stimulation pulse 504. As illustrated in FIG. 5, stimulation interference signals 509 and ECAPs 508 may be recorded on channel 506.

In some examples, IMD 200, for example, may deliver the entire group of stimulation pulses 504 (e.g., a sweep) consecutively and without any other intervening pulses in order to detect ECAPs 508 from which respective characteristic values are determined. IMD 200 may then determine the relationship between the characteristic values from ECAPs 508 and the different parameter values of stimulation pulses 504. In one example, the sweep of pulses 504 may be delivered by IMD 200 during a break in delivery of other types of stimulation pulses.

As described herein, IMD 200 may deliver stimulation configured to selectively activate smaller nerve fibers instead of larger fibers in order to promote an anesthesia effect over a paresthesia effect. For example, IMD 200 may deliver a burst of pulses that condition larger nerve fibers by increasing the activation threshold for those larger nerve fibers. This burst of pulses may have sub-perception threshold intensity such that the patient does not perceive the stimulation, but in some examples the amplitude of the burst of pulses may be low but still above perception threshold. The burst of pulses may be delivered prior to a stimulation pulse that then activates more smaller nerve fibers and fewer larger nerve fibers as a result of the activation threshold of the larger nerve fibers being increased by the burst of pulses.

IMD 200 can then assess why types of nerves are being activated by analyzing ECAP signals elicited by the stimulation pulses. For example, IMD 200 may analyze latencies in the form of time for peaks to occur from delivery of the stimulation pulse, peak-to-peak durations, peak amplitudes, or frequency content in the ECAP signal. For example, longer latencies in the ECAP signal indicate that more small fibers are activating than large fibers. In some examples, the burst of pulses may be delivered with a passive recharge pulse or active recharge pulse, depending on system power availability and/or time duration available between pulses for passive recharge to occur. In some examples, IMD 200 may receive patient input indicative of the patient's perception of paresthesia and pain relief. A higher perception of paresthesia may be indicative of more large fiber activation, and IMD 200 my respond by adjusting one or more stimulation parameter values that define the burst of pulses in order to increase the activation threshold of the larger fibers for subsequent stimulation pulses.

FIG. 6A is a graph of example differential ECAP curves indicative of different fiber latencies at different amplitude values. As shown in FIG. 6A, graph 600 illustrates different stimulation pulses 602 that elicit respective ECAP signals 608 and differential ECAP curves 610. IMD 200 may deliver different stimulation pulses at different amplitudes and receive information representative of the plurality of ECAP signals 608 elicited by the different stimulation pulses defined by different parameter values. As shown in FIG. 6A, the stimulation pulses differ by amplitude, ranging from 0.8 mA up to 2.8 mA.

IMD 200 may then determine, based on the plurality of ECAP signals 608, a differential ECAP characteristic indicative of the latency of a plurality of different fiber types activated by the different stimulation pulses. The differential ECAP characteristic may be differential ECAP curves 610 or another characteristic determined based on the differential ECAP curves 610 or other aspect of ECAP signals 608. IMD 200 may then determine, based on the differential ECAP characteristic, at least one parameter value of the parameter set defining the stimulation pulse and determine, based on the differential ECAP characteristic, at least one parameter value of the parameter set defining the burst of pulses that will precede the stimulation pulse.

In the example of graph 600, sections 604 and 606 correspond to peaks having different latencies because of where the peaks are located. Section 604 may correspond to peaks resulting from larger nerve fibers (e.g., type 1 fibers) and second 606 may correspond to peaks resulting from smaller nerve fibers (e.g., type 2 fibers) that have a higher latency when compared to type 1 fibers. IMD 200 can decompose the individual ECAP signals 608 to determine differential ECAP curves 610. In particular, differential ECAP curves 610 indicate where the larger proportion of the peaks are occurring. At amplitude 1.2, the largest amplitude of differential ECAP curve 610 occurs for type 1 fibers within section 604. The largest amplitude of differential ECAP curves 610 occurs for type 2 fibers within section 606 at amplitude 2.2 mA. Based on the differential ECAP curves, IMD 200 may thus set the amplitude for the stimulation pulse at or above the type 2 threshold of 2.2 mA because that increases the type 2 fibers being activated. IMD 200 can then determine the conditioning pulses (e.g., the burst of pulses) parameters to minimize the type 1 response in ECAP signals.

In some examples, IMD 200 may cycle the bursts of pulses on and off to reduce energy consumption. Since the conditioning effect for type 1 fibers may last longer than the intervals between stimulation pulses, IMD 200 may not need to deliver the busts of pulses until the condition effect has worn off or is no longer sufficient to reduce the type 1 response. In some examples, IMD 200 may monitor the posture of the patient and increase or decrease the amplitude of the stimulation pulses to maintain the type 2 response while reducing the type 1 response. In other words, IMD 200 may have different parameter values for different postures. In some examples, IMD 200 may monitor changes to ECAP amplitudes and adjust the amplitudes of stimulation pulses and/or bursts of pulses to maintain the desired effects.

FIG. 6B is a graph of differential ECAP characteristics representative of the differential ECAP curves in FIG. 6A. As shown in the example of FIG. 6B, graph 612 includes response 614 for type 1 fibers and response 616 for type 2 fibers. The differential ECAP characteristic, or differential latency, may be the difference between each of responses 614 and 616 at each amplitude level on the x-axis. In other words, each of responses 614 and 616 are the maximum ECAP signal amplitudes for each type of fiber at each stimulation pulse amplitude. This differential ECAP characteristic represents the relative difference between type 1 fiber activation and type 2 fiber activation. Therefore, the greatest ratio of type 1 response to type 2 response occurs at 1.2 mA, and the greatest ratio of type 2 response to type 1 response occurs at 2.2 mA.

FIG. 6C includes graphs of different ECAP characteristic values for different parameters defining burst of pulses and stimulation pulses. As shown in FIG. 6C, graphs 620A-620H (collectively “graphs 620) are example graphs of different ECAP signal responses to a stimulation pulse that follows bursts of pulses having different parameter values. The different parameters for the bursts of pulses include different number of pulses in each burst, whether the recharge pulse was active or passive for each pulse of the burst, and different amplitudes of the pulses in each burst.

As shown in the example of graphs 620, amplitudes of the bursts of pulses are shown as a fraction of the perception threshold. Therefore, amplitude values over 1.0 are supra-threshold and elicit an ECAP response after each pulse in the burst. Amplitude values at 1.0 or below are sub-threshold pulses and are not perceptible to the patient. Therefore, curves with lower ECAP amplitudes are preferred since the patient does not perceive delivery of the bursts of pulses and the ECAP signal elicited after the stimulation pulse also is very low and indicates a lack of patient perception. In the example of FIG. 6C, graph 620E illustrates a preferred parameter set with a burst of pulses having 5 pulses that have passive recharge and an amplitude set to 80% of perception threshold. These values may be different for different patients. In some examples, the system may automatically estimate the perception threshold by generating a growth curve of ECAP amplitudes from a sweep of multiple stimulation pulses and identifying an inflection point in the growth curve or other stimulation amplitude or ECAP amplitude at which the stimulation pulses likely are starting to be perceptible to the patient.

FIGS. 6D and 6E are graphs of different nerve fiber locations with respect to the midline of the spinal cord. In addition to determining appropriate parameter values for effective therapy, latency from ECAP signals may also be used by system 100 to determine where to implant electrodes or determine which electrodes to use for delivering stimulation. As shown in FIGS. 6D and 6E, the distribution of nerve fiber types varies by distance from the midline of the spinal cord, with the higher percentage of larger and faster fibers located about 1.5 mm-2.0 mm lateral from the midline. Therefore, electrodes located at the midline of the spinal cord will result in exiting slower fibers first at lower amplitudes and then excite faster fibers also at higher amplitudes. Therefore, system 100 can use this information to determine where the electrodes are located. When attempting to stimulate slower fibers, electrode location closer to the midline of the spinal cord may result in stimulation of smaller fibers instead of larger fibers.

In one example, the clinician or system 100 may determine where to implant electrodes or which electrodes to deliver. Alternatively, after lead implantation, the system 100 may similarly obtain ECAP signals elicited by different electrode combinations and select the electrode combination for subsequent stimulation based on the latency of the ECAP signals from the respective electrode combinations. In one example, system 100 may control delivery of a stimulation pulse via one or more electrodes and receive information representative of an evoked compound action potential (ECAP) signal elicited by the stimulation pulse. System 100 can then determine an ECAP characteristic of the ECAP signal, the ECAP characteristic indicative of a latency of one of more nerve fibers activated by the stimulation pulse, and determine, based on the ECAP characteristic indicative of the latency, an implant location for the one or more electrodes. In some examples, system 100 may perform this process over many ECAP signals from many stimulation pulses. For example, system 100 may determine that a second latency is shorter than a first latency determined from a precious ECAP signal and then determine, based on the second latency being shorter than the first latency, that the one or more electrodes having moved laterally from a midline of a spinal cord. Given the desired pain coverage, e.g. with pain being bilateral, or coming from more caudal parts of the body (e.g. back and legs for thoracic stimulation in region from T7 to T9), it may be useful to deliver stimulation pulses closer to midline of the spinal cord than lateral regions. For these applications, the system may be adjusted based on ECAP latency to select stimulation parameter values (e.g., including electrode combinations) that elicit relatively longer latencies from a greater percentage of smaller fibers being activated. Alternatively, several electrodes may provide current as part of a single electrode combination (e.g. current steering) to steer current across the electrode array to target the stimulation location to the appropriate anatomical target based on ECAP latency. For example, if it is determined that one lead (when delivering stimulation by itself) excites nerve fibers laterally on the left side, while the other lead delivers stimulation on the right side, then steering between the left side and the right side may be utilized to effectively provide stimulation, or activation of nerves, at the most appropriate midline location. Specifically, the weighting of the excitation between the two leads (e.g., the proportion of current delivered from electrodes of the respective lead) may be adjusted to achieve a desired therapeutic effect by using the latency of the excitation.

FIGS. 6F and 6G are graphs representing different ECAP signals from different stimulation amplitudes. As shown in FIG. 6F, the graph of ECAP signals from respective stimulation pulses at different amplitudes illustrates that large fibers at higher amplitudes produce peaks with less latency. Conversely, lower amplitude stimulation pulses result in peaks having longer latencies as a result of smaller fibers being activated. FIG. 6G illustrates an example of the relative stimulation amplitude on the y-axis vs. the latency on the x-axis. At lower relative stimulation amplitudes, the latency is longer for the ECAP signal.

In some examples, latency (or other indication of fiber type activity) may be an ECAP characteristic that is determined from the timing of a peak amplitude. In other examples, as shown in FIGS. 6H-6L, the latency of an ECAP signal (e.g., an ECAP characteristic) can be determined from a principal component analysis (PCA) decomposition of the ECAP signal. In this manner, the ECAP signal may be processed to determine the latency and indicator of the fibers activated from the stimulus. In other words, the system can apply the PCA process to determine the type of fibers representative of the ECAP signal, of which latency can be representative of the type of fibers. In some examples, the amplitudes of each ECAP signals may be normalized to calculate the latency. Although PCA is described herein, other methods such as advancement reduction or machine learning can also be used to determine the latency of ECAP signals and which type of fibers are more representative of the fibers activated by the stimulus. The PCA can also be beneficial to signal analysis by removing noise from the signals, and may be more robust analysis of fiber type than just using peak-to-peak latency values.

FIGS. 6H and 6I are graphs representing different components of ECAP signals using a principle component analysis (PCA). As shown in the example of FIG. 6H, the four largest components of ECAP signals are graphed. Each component may represent variables in the data that have different variability, with the first component having the most variability. As shown in FIG. 61, the first few components account for the vast majority of the percentage of the shape of the ECAP signals. Therefore, these first components may be the most appropriate for identifying differences between ECAP signals.

FIG. 6J provides graphs of correlation of different components of a PCA of ECAP signals. As can be seen in graphs 642, 644, and 646, there is no correlation between the combinations of components 1 and 3, 1 and 4, or 1 and 5. However, graph 640 illustrates that there is high correlation between the first and second components in the form of a semi-circular pattern in the correlation. Therefore, the system may use the correlation of the first and second components to identify the variability of latency between different ECAP signals.

The processing circuitry of the system, such as processing circuitry 208, may determine the ECAP type (or indication of latency) via a processing technique that leverages PCA. For example, processing circuitry 208 can average the ECAP signals, which can be during the aggressor (e.g., the patient movement or at rest). Processing circuitry 208 can then subtract any stimulation artifacts, such as via an exponential model or filtering and then the exponential model, and normalize the amplitudes of the signals to each other. Next, processing circuitry 208 can perform the PCA to identify the dominant components. In some examples, these components may be referred to as weights or other values indicative of the signals. In some examples, the dominant components may be utilized from previously analyzed data from the same patient or other patients. Processing circuitry 208 can then identify the ECAP type (or latency) based on the PCA analysis. For example, processing circuitry 208 can translate the PCA components into a continuum as shown to determine Theta or other variable, and then output the ECAP type. In some examples, processing circuitry 208 can perform an action based on the type of ECAP (or fibers), such as determine an appropriate placement of a lead, identify lead migration, or adjust one or more stimulation parameter values during closed-loop feedback control of therapy.

In some examples, this process can be used to determine whether the signal includes or does not include an ECAP signal, determine the type of fibers activated (of which latency may be representative), determine lead position or lead migration, therapy efficacy, therapy changes, patient responder type, or any other information associated with the patient and/or therapy.

FIG. 6K is a graph illustrating example latencies presented as theta of different ECAP signals and example indications that can be presented. As shown in the example of FIG. 6K, each data point 650 in the graph indicates the value of the first and second component correlation for each ECAP signal from the sample signals. The resulting correlation curve 652 is generally semi-circular. Once this correlation curve 652 is determined, subsequent ECAP signals can be processed and a latency determined for the ECAP signal. Angle 654 may represent the angle Theta that is indicative of the latency. Smaller angles, or smaller Theta, occurring on the left side of correlation curve 652 may indicate smaller latencies. In a user interface, for example, this data in this graph, or some portion of it, can be displayed. For example, arrow 658 may point to the latency of the latest ECAP signal or the signal of interest. In some examples, text box 656 may provide the value of Theta, the latency value, or other latency related value, for a specific ECAP signal which may correspond to the fiber types activated by the stimulus that elicited the ECAP signal. In some examples, the system may establish ranges of Theta that correspond to different types of fibers that make up most of the activation from the stimulus.

FIG. 6L provides graphs of example ECAP signals and theta values representative of latency for that ECAP signal. As shown in the examples of FIG. 6L, theta is greater for those ECAP signals that have larger latencies, and theta is less for those ECAP signals with less latencies.

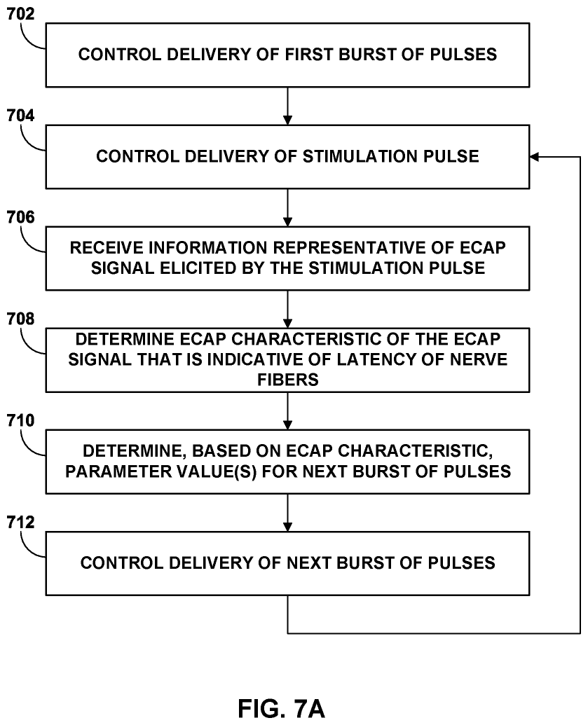

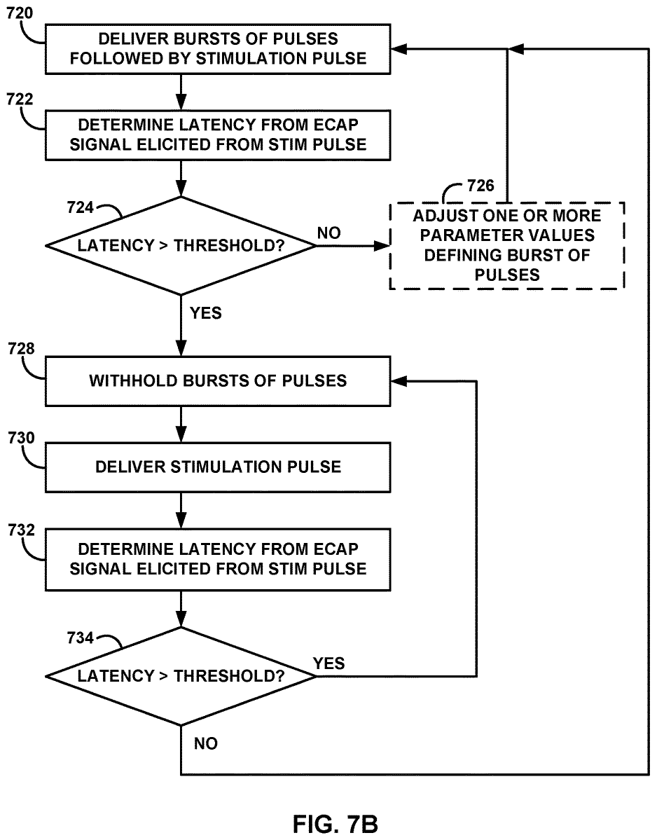

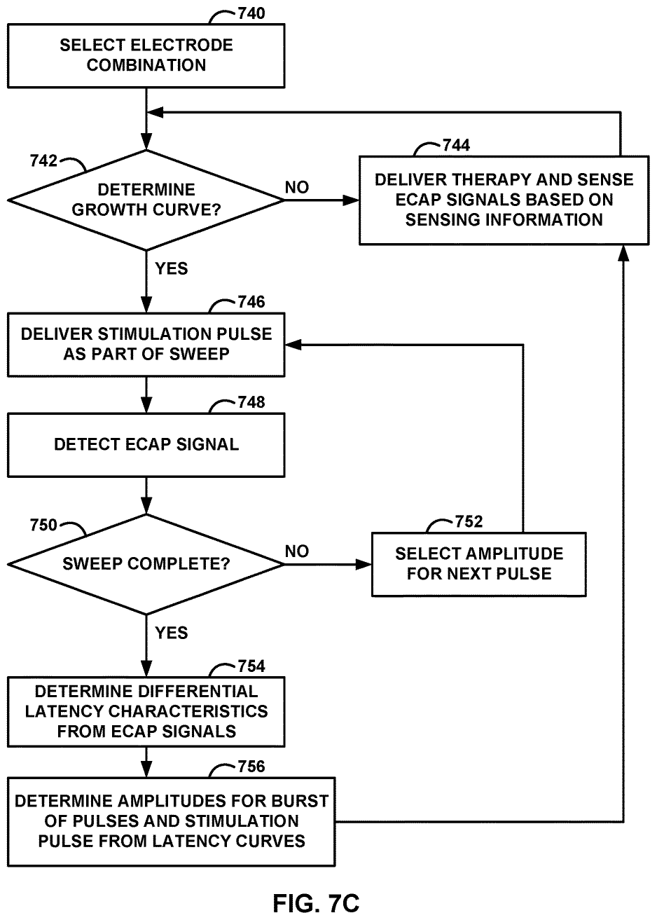

FIG. 7A is a flow diagram illustrating an example technique for adjusting parameter values based on an indication of latency in ECAP signals. For convenience, FIG. 7A is described with respect to IMD 200 of FIG. 2. However, the techniques of FIG. 7A may be performed by different components of IMD 200 or by additional or alternative medical devices. FIG. 7A will be described using bursts of pulses and stimulation pulses that eliciting detectable ECAP signals. Although processing circuitry 208 will be described as performing much of the technique of FIG. 7A, other components of IMD 200 and/or other devices may perform some or all of the technique in other examples.

In the example operation of FIG. 7A, processing circuitry 208 controls stimulation generation circuitry 204 to deliver a first burst of pulses (702). As described herein, the burst of pulses may be conditioning pulses configured to increase the activation threshold for a first type of nerve fibers such that subsequent stimulation does not activate that first type of nerve fibers. Then, processing circuitry 208 controls stimulation generation circuitry 204 to deliver a stimulation pulse (704). Processing circuitry 208 may deliver the stimulation pulse a predetermined amount of time after the burst of pulses has been delivered.

Next, processing circuitry 208 receives information representative of the ECAP signal elicited by the stimulation pulse (706). Sensing circuitry 206 may sense the ECAP signal. Processing circuitry 208 next determines an ECAP characteristic of the ECAP signal that is indicative of the latency of nerve fibers (708). This ECAP characteristic may include a latency of one or more peaks in the ECAP signal, a differential latency of peaks in the signal corresponding to different fiber types, or any other such information. In some examples, the latency may be determined using the PCA technique described herein. Based on the ECAP characteristic, processing circuitry 208 then determines parameter values for the next burst of pulses (710). In some examples, processing circuitry 208 may adjust one or more parameters to different respective values if the latency is below threshold or otherwise indicates that the first fiber type is not being effectively conditions. In examples in which the latency is above threshold or otherwise indicative of effective small fiber activation, processing circuitry 208 may not change any stimulation parameter values for the next burst of pulses. Using the determine stimulation parameters, processing circuitry 208 may then control delivery of the next burst of pulses (712) before controlling delivery of the next stimulation pulse (704). This process may continue in a closed-loop manner to control stimulation.

Although generally described as sensing ECAP signals elicited by the stimulation pulse that occurs after the bust of pulses, in other examples, the system may sense one or more ECAP signals elicited by the burst of pulses. The system may similarly analyze the ECAP signal for ECAP characteristics such as latency, and determine or adjust stimulation parameters or feedback variables based on the ECAP characteristic identified from the ECAP signal elicited by the burst of pulses.

As described herein, adjustments to stimulation parameters based on an ECAP latency may be a specific selected value. In other examples, the system may deliver a plurality of subsequent pulses having a random, pseudorandom, or stochastic variation in the parameter values between the pulses and analyze the respective ECAP signals for ECAP latencies and identify the parameter value(s) that elicited the desired ECAP latency. In other examples, a parameter, such as amplitude, or the burst of pulses or the single stimulation pulses delivered after each burst of pulses may be varied over the course of time in a random, pseudorandom, or stochastic variation in order to achieve a desired percentage or critical mass of ECAP latencies within a desired range. In some examples, determinations or adjustments to parameter values may be made based on averages, moving averages, or other collective sensed value.

FIG. 7B is a flow diagram illustrating an example technique for cycling the delivery of conditioning bursts of pulses while continuing to deliver stimulation pulses. For convenience, FIG. 7B is described with respect to IMD 200 of FIG. 2. However, the techniques of FIG. 7B may be performed by different components of IMD 200 or by additional or alternative medical devices. FIG. 7B will be described using bursts of pulses and stimulation pulses that eliciting detectable ECAP signals. Although processing circuitry 208 will be described as performing much of the technique of FIG. 7B, other components of IMD 200 and/or other devices may perform some or all of the technique in other examples.

As shown in the example of FIG. 7B, processing circuitry 208 may control delivery of a burst of pulses followed by a stimulation pulse (720). Processing circuitry 208 then determines a latency from the ECAP signal elicited by the stimulation pulse (722). The latency may be any type of ECAP characteristic associated with the latency of one or more aspects of the ECAP signal, such as the latency to the N1 peak associated with the smaller fibers or the differential latency between the different peaks in the signal. If the latency is not greater than a latency threshold (“NO” branch of block 724), processing circuitry 208 may continue to deliver bursts of pulses (720). In some examples, processing circuitry 208 may adjust one or more parameter values that defines the bursts of pulses in an attempt to increase the latency of the ECAP signal and increase the activation of smaller fibers instead of larger fibers (726).

If the latency is greater than the threshold (“YES” branch of block 724), processing circuitry 208 withholds bursts of pulses (i.e., does not deliver an otherwise scheduled burst of pulses) (728) before then delivering the stimulation pulse 730). In this manner, processing circuitry 208 stops delivery of the bursts of pulses. Processing circuitry 208 then determines the latency from the ECAP signal elicited from the stimulation pulse (732) before again determining if the latency is greater than the latency threshold (734). If the latency is greater than the threshold (“YES” branch of block 734), processing circuitry 208 continues to withhold the bursts of pulses (728). If the latency is not greater than a latency threshold (“NO” branch of block 734), processing circuitry 208 cycles the bursts back on and restarts delivering bursts of pulses (720).

FIG. 7C is a flow diagram illustrating an example technique for determining differential latency characteristics from ECAP signals, in accordance with one or more techniques of this disclosure. For convenience, FIG. 7C is described with respect to IMD 200 of FIG. 2. However, the techniques of FIG. 7C may be performed by different components of IMD 200 or by additional or alternative medical devices. IMD 200, for example, may use detected ECAP signals to determine latency curves from which a differential latency can be determined (as described with respect to FIGS. 6A and 6B). Although processing circuitry 208 will be described as performing much of the technique of FIG. 7C, other components of IMD 200 and/or other devices may perform some or all of the technique in other examples.

In the example operation of FIG. 7C, processing circuitry 208 selects the electrode combination for delivery of stimulation pulses (740). This selection may be automatic or at least partially in response to user selection of one or more electrodes. Processing circuitry 208 may then determine whether or not new growth curves for nerve fibers need to be determined or calibrated (742). If processing circuitry 208 does not have instructions to determine growth curves, such as to calibrate or recalibrate a latency threshold or determine parameters (“NO” branch of block 742), processing circuitry 208 may continue to deliver stimulation and detect ECAP features according to the sensing parameters stored in memory (744).

If processing circuitry 208 does have instructions to determine growth curves for latency determinations (“YES” branch of block 742), processing circuitry 208 controls stimulation circuitry 202 to deliver the first stimulation pulse as part of a sweep of pulses with different parameter values (746). Processing circuitry 208 controls sensing circuitry 206 to detect the ECAP signal elicited by the stimulation pulse (748). If the sweep is not complete (e.g., there are more pulses of the sweep to be delivered) (“NO” branch of block 750), processing circuitry 208 selects the next stimulation parameter value (e.g., the next current amplitude) for the next stimulation pulse of the sweep (752) and controls stimulation circuitry 202 to deliver the next stimulation pulse of the sweep (746). A sweep of stimulation pulses may include at least two pulses, four or more pulses, or six or more pulses. In addition, the sweep may only increase the stimulation parameter value, only decrease the stimulation parameter value, or perform iterative increases in the stimulation parameter value and iterative decreases in the stimulation parameter value. Although more pulses may enable a more accurate relationship, as few pulses as possible may be used to reduce the amount of time needed to deliver pulses of the sweep and sense the resulting ECAP signals. In some examples, processing circuitry 208 may complete these sweeps for some or all posture states of the patient in order to determine relationships for each posture state.

If the sweep is complete and there are no more stimulation pulses of the sweep to be delivered (“YES” branch of block 750), processing circuitry 208 analyzes the detected ECAP signals from the sweep and determines one or more latency curves for these detected ECAP signals and/or differential latency characteristics from the ECAP signals (754). The analysis of the detected ECAP signals may include determining the latency, or delay, or one or more features of the ECAP signal to the delivery of the stimulation pulse (e.g., the latency of the N1 peak or some other feature) and then associating that latency value to at least one parameter value (e.g., pulse current amplitude) that defined the stimulation pulse that elicited the characteristic value. In other examples, the differential latency characteristic may include the differential latency curves like in FIG. 6A from which the differential latency characteristics can be determined. All of the latency values and associated parameter values can be plotted, and processing circuitry 208 may determine a best fit line to the points and determine the slope of that best fit line for a particular latency curve. In other examples, processing circuitry 208 may determine a relationship between the latency value and respective parameter values that is different than a latency curve. In some examples, processing circuitry 208 may determine one latency curve for increasing the stimulation parameter value and another latency curve for decreasing the stimulation parameter value. In this manner, the relative proportion of different types of fibers that are activated may be affected by whether or not stimulation amplitudes are increasing or decreasing with respect to previous stimulation pulses.

Processing circuitry 208 may then determine the amplitudes (or other parameter values) for bursts of pulses and the stimulation pulses based on the latency curves (756). For example, processing circuitry 208 may determine at which pulse amplitudes the differential latency is greatest for different fiber types as described with respect to FIGS. 6A and 6B. In other examples, processing circuitry 208 may identify the amplitude for which desired fiber type responses are present in the ECAP signals or latency curves. Processing circuitry 208 may store these latency characteristics and determined parameter values in memory 216. Processing circuitry 208 may then use the parameter values and/or latency characteristics or thresholds as part of the therapy parameters and closed-loop criteria in subsequent stimulation (744). Processing circuitry 208 may perform the calibration process of FIG. 7C during initial set up and programming of IMD 200 and, in some examples, periodically during therapy as the patient's sensitivity to stimulation pulses may change over time. Processing circuitry 208 may request recalibration of the latency curves in response to determining that ECAP characteristic values are not expected or therapy is no longer efficacious. Alternatively, a user may request, via a user interface, that processing circuitry 208 recalibrate the latency curves or parameter values if closed-loop control of stimulation therapy is no longer effective.

As described herein, the ECAP signal sensed may be the ECAP from tissue that was the target tissue for the delivered stimulus. In other examples, the ECAP signal sensed may be downstream on nerve fibers different than the original nerve fibers activated by the stimulus. In this manner, the ECAP signal may be sensed from a neural circuit different than the original circuit activated by the stimulus. In one examples, stimulation of the thalamus may result in an ECAP sensed at the cortex of the patient. In another example, a stimulus applied to the dorsal column may result in an ECAP recorded at the scalp or at a peripheral nerve. In this manner, the system described herein may deliver stimulus to a peripheral nerve and sense ECAP signals at the dorsal column or dorsal root, for example, or stimulate and sense the ECAP signal at the same or different peripheral nerves. In any event, as described herein, an ECAP signal may be recorded by electrodes on the same lead that delivered the stimulus or recorded by electrodes on a different lead than the stimulation electrodes.

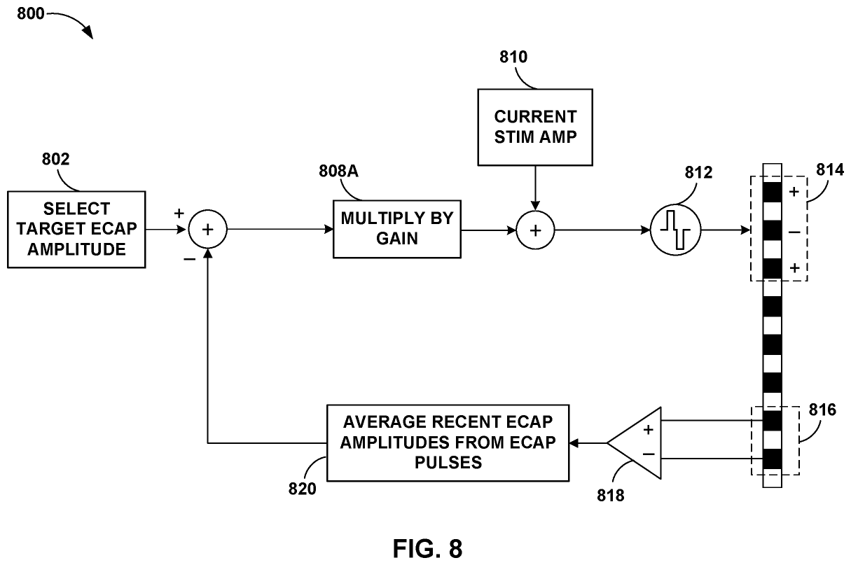

FIG. 8 is a diagram illustrating an example technique 800 for adjusting stimulation therapy. As shown in the example of FIG. 8, the system, such as IMD 200 or any other device or system described herein, may dynamically adjust pulse amplitude (or other parameter) based on the gain value representing the patient sensitivity to stimulation. IMD 200 may perform this process instead of, or in addition to, adjusting parameter values based on latency characteristics as described herein. Processing circuitry 208 of IMD 200 may control stimulation generation circuitry 204 to deliver a stimulation pulse to a patient. Processing circuitry 208 may then control sensing circuity 206 to sense an ECAP signal elicited by the pulse and then identify a characteristic of the ECAP signal (e.g., an amplitude of the ECAP signal). Processing circuitry 208 may then control stimulation generation circuitry 204 to deliver the stimulation pulse according to the determined stimulation pulse.

As shown in the example of FIG. 8, a pulse 812 is delivered to the patient via electrode combination 814, shown as a guarded cathode of three electrodes. The resulting ECAP is sensed by the two electrodes at the opposing end of the lead of electrode combination 816 fed to a differential amplifier 818. For each sensed ECAP, processing circuitry 208 may measure an amplitude of a portion of the ECAP signal based one or more features within respective sensing windows, such as the N1-P2 voltage amplitude from the portion of the ECAP signal. Processing circuitry 208 may average the recently measured ECAP amplitudes, such as averaging the most recent, and consecutive, 2, 3, 4, 5, 6, or more ECAP amplitudes. In some examples, the average may be a mean or median value. In some examples, one or more ECAP amplitudes may be ignored from the calculations if the amplitude value is determined to be an error. The measured amplitude 820 (or average measured amplitude) is then subtracted from the selected target ECAP amplitude 802 to generate a differential amplitude (e.g., an ECAP differential value). The selected target ECAP amplitude 802 may be determined from an ECAP sensed when the physician or patient initially discovers effective therapy from the stimulation pulses. This target ECAP amplitude 802 may essentially represent a reference distance between the stimulation electrodes and the target neurons (e.g., the spinal cord for the case of SCS). The target ECAP amplitude 802 may also represent the target neural recruitment for the patient.

The differential amplitude may represent whether the stimulation intensity of the next stimulation pulse should increase or decrease in order to achieve the target ECAP amplitude 802. For example, a positive differential amplitude indicates that the measured amplitude (e.g., the determined characteristic value of the last one or more ECAP signals) is less than the target ECAP amplitude 802 and the stimulation intensity needs to increase in order to increase neural recruitment to achieve neural recruitment closer to the ECAP amplitude 802. Conversely, a negative differential amplitude indicates that the measured amplitude (e.g., the determined characteristic value of the last one or more ECAP signals) is greater than the target ECAP amplitude 802 and the stimulation intensity needs to decrease in order to decrease neural recruitment to achieve neural recruitment closer to the ECAP amplitude 802.

The differential amplitude is then multiplied by the gain value for the patient to generate a differential value 808A. Processing circuitry 208 may add the differential value 808A to the current pulse amplitude 810 to generate the new, or adjusted, pulse amplitude that at least partially defines the next pulse 812.

The following formulas may represent the function used to calculate the pulse amplitude of the next pulse 812. Equation 1 below represents an equation for calculating the new current amplitude using a linear function, wherein Ac is the current pulse amplitude, D is the differential amplitude by subtracting the measured amplitude from the target ECAP amplitude, G is a real number for the gain value, and AN is the new pulse amplitude:

A_{N}=A_{C}+(D×G) (1)

In some examples, the gain value G is a constant for increasing stimulation intensity or decreasing stimulation intensity. In this manner, the gain value G may not change for a given input. It is noted that different gain values may be employed for increasing stimulation than decreasing stimulation, as discussed herein. Alternatively, processing circuitry 208 may calculate the gain value G such that the gain value varies according to one or more inputs or factors. In this manner, for a given input or set of inputs, processing circuitry 208 may change the gain value G. Equation 2 below represents an example linear function for calculating the gain value, wherein M is a multiplier, D is the differential amplitude by subtracting the measured amplitude from the target ECAP amplitude, and G is the gain value:

G=M×D (2)

Processing circuitry 208 may use the gain value G calculated in Equation 2 in Equation 1. This would result in Equation 1 being a non-linear function for determining the new current amplitude. According to Equation 2 above, the gain value G may be greater for larger differences between the measured amplitude and the target ECAP amplitude. Thus, gain value G will cause non-linear changes to the current amplitude. In this manner, the rate of change in the current amplitude will be higher for larger differences between the measured amplitude and the target ECAP amplitude and lower for smaller differences between the measured amplitude and the target ECAP amplitude. In other examples, a non-linear function may be used to calculate the gain value G.

The stimulation pulse may be a monophasic pulse followed a passive recharge phase. However, in other examples, the pulse may be a bi-phasic pulse that includes a positive phase and a negative phase. In some examples, a pulse may be less than 300 μs, but the following passive recharge phase or even an active recharge phase (of a bi-phasic pulse) may still obscure the detectable ECAP signal from that pulse. In other examples, the pulse width of the stimulation pulse may be greater than 300 μs, but some of the ECAP signal may be obscured by the stimulation pulse.

In some examples, depending upon, at least in part, pulse width of the stimulation pulse, IMD 110 may not sufficiently detect an ECAP signal because the stimulation pulse is also detected as an artifact that obscures the ECAP signal. If ECAPs are not adequately recorded, then ECAPs arriving at IMD 110 cannot be used to determine the efficacy of stimulation parameter settings, and electrical stimulation signals cannot be altered according to responsive ECAPs. In some examples, pulse widths may be less than approximately 300 μs, which may increase the amount of each ECAP signal that is detectable. Similarly, high pulse frequencies may interfere with IMD 110 sufficiently detecting ECAP signals. For example, at pulse frequency values (e.g., greater than 200 Hz, greater than 100 Hz, etc.) that cause IMD 110 to deliver another pulse before an ECAP from the previous pulse can be detected, IMD 110 may not be capable to detecting the ECAP.

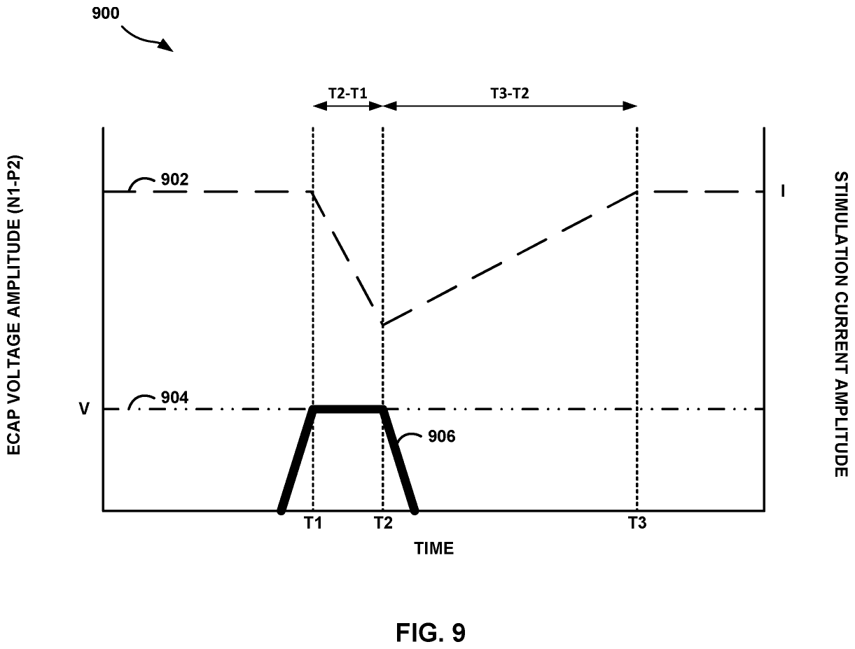

FIG. 9 illustrates a graph 900 that includes pulse current amplitude 902, threshold ECAP amplitude 904 (e.g., a type of threshold ECAP characteristic value), and sensed ECAP voltage amplitude 906 as a function of time, in accordance with one or more techniques of this disclosure. For convenience, FIG. 9 is described with respect to IMD 200 of FIG. 2. However, the techniques of FIG. 9 may be performed by different components of IMD 200 than as described herein or by additional or alternative medical devices. IMD 200 may perform the techniques of FIG. 9 in additional to adjusting parameter values of the bursts of pulses and/or the stimulation pulses based on latency characteristics.

Graph 900 illustrates a relationship between sensed ECAP voltage amplitude and stimulation pulse current amplitude. For example, pulse current amplitude 902 is plotted alongside ECAP voltage amplitude 906 as a function of time, showing how processing circuitry 208 can change stimulation current amplitude relative to ECAP voltage amplitude. In some examples, IMD 200 delivers a plurality of pulses at pulse current amplitude 902. Initially, IMD 200 may deliver a first set of stimulation pulses at current amplitude I. The first set of stimulation pulses may be delivered prior to time T1. In some examples, current amplitude I is less than 25 milliamps (mA) and can be between about 2 mA and about 18 mA. However, current amplitude I may be any current amplitude that IMD 200 can deliver to the patient and appropriate for effective stimulation therapy for the patient.

While delivering the first set pulses, IMD 200 may record ECAP voltage amplitude 906 from ECAPs elicited from the respective pulses. During transient patient movement, ECAP voltage amplitude 906 may increase if pulse current amplitude 902 is held constant and the distance between the electrodes and target nerve decreases. For example, as illustrated in FIG. 9, ECAP voltage amplitude 906 may increase prior to time T1 while stimulation current amplitude is held constant. An increasing ECAP voltage amplitude 906 may indicate that patient 102 is at risk of experiencing transient overstimulation due to the pulses delivered by IMD 200. To prevent patient 102 from experiencing transient overstimulation, IMD 200 may decrease pulse current amplitude 902 in response to ECAP voltage amplitude 906 exceeding the threshold ECAP amplitude 904. For example, if IMD 200 senses an ECAP having an ECAP voltage amplitude 906 meeting or exceeding threshold ECAP amplitude 904, as illustrated in FIG. 9 at time T1, IMD 200 may enter a decrement mode where pulse current amplitude 902 is decreased. In some examples, the threshold ECAP amplitude 904 is greater than 10 microvolts (μV) and less than 100 μV. For example, the threshold ECAP amplitude 904 can be 30 μV. In other examples, the threshold ECAP amplitude 904 is less than or equal to 10 μV or greater than or equal to 100 μV. The exact value of threshold ECAP amplitude 904 may depend on the patient's perception of the delivered stimulation, as well as the spacing between the sensing/stimulation electrodes and the neural tissue, whether or not stimulation intensity is increasing or decreasing, or other factors.

The decrement mode with a plurality of decrement rate settings may, in some cases, be stored in memory 216 of IMD 200 as a part of stimulation parameter settings 220. In the example illustrated in FIG. 9, the decrement mode is executed by IMD 200 over a second set of pulses which occur between time T1 and time T2. In some examples, to execute the decrement mode, IMD 200 decreases the pulse current amplitude 902 of each pulse of the second set of pulses according to a first linear function with respect to time. During a period of time in which IMD 200 is operating in the decrement mode (e.g., time interval T2−T1), ECAP voltage amplitude 906 of ECAPs sensed by IMD 200 may be greater than or equal to threshold ECAP amplitude 904.

In the example illustrated in FIG. 2, IMD 200 may sense an ECAP at time T2, where the ECAP has an ECAP voltage amplitude 906 that is less than threshold ECAP amplitude 904. The ECAP sensed at time T2 may, in some cases, be the first ECAP sensed by IMD 200 with a below-threshold amplitude since IMD 200 began the decrement mode at time T1. Based on sensing the ECAP at time T2, IMD 200 may deactivate the decrement mode and activate an increment mode. As discussed herein, IMD 200 may use a gain value selected for the increment mode such that the magnitude of the increase in stimulation parameter is appropriate for increasing the stimulation intensity of the next stimulation pulses. The increment mode with a plurality of increment rate settings may, in some cases, be stored in memory 216 of IMD 200 as a part of stimulation parameter settings 220. IMD 200 may execute the increment mode over a third set of pulses which occur between time T2 and time T3. In some examples, to execute the increment mode, IMD 200 increases the pulse current amplitude 902 of each pulse of the third set of pulses according to a second linear function with respect to time, back up to the initial current amplitude I that may be predetermined for therapy. In other words, IMD 200 increases each consecutive pulse of the third set of pulses proportionally to an amount of time elapsed since a previous pulse. Although IMD 200 may increase and decrease the amplitudes by linear functions in some examples, IMD 200 may employ non-linear functions in other examples. For example, the gain value may represent a non-linear function in which the increment or decrement changes exponentially or logarithmically according to the difference between the sensed ECAP characteristic value and the threshold ECAP amplitude 904.

When pulse current amplitude 902 returns to current amplitude I (e.g., the predetermined value for stimulation pulses), IMD 200 may deactivate the increment mode and deliver stimulation pulses at constant current amplitudes. By decreasing stimulation in response to ECAP amplitudes exceeding a threshold ECAP characteristic value and subsequently increasing stimulation in response to ECAP amplitudes falling below the threshold, IMD 200 may prevent patient 102 from experiencing transient overstimulation or decrease a severity and/or a time duration of transient overstimulation experienced by patient 102. In some examples, threshold ECAP amplitude 904 may include an upper threshold and a lower threshold, such that IMD 200 enters the decrement mode when the upper threshold is exceeded, IMD 200 enters the increment mode when the lower threshold is exceeded, and IMD 200 maintains stimulation parameter values when ECAP voltage amplitude 906 is between the upper threshold and the lower threshold.

In some examples, the system may be configured to detect or identify lead migration based on changes in ECAP latency over time. For example, the ECAP latency can be stored in memory 216 of IMD 200, and, over a duration of hours, weeks, months, or years, if the latency differs more than a threshold amount, the system may trigger a warning of potential lead migration. In another example, if the relationship between sensed ECAP voltage amplitude and stimulation pulse current amplitude changes beyond a threshold value, the system may trigger a warning. In response to the warning, the system may operate to obtain new growth curves and/or adjust patient programming. The system may display the ECAP latency values, or changes, over time for a user. In some examples, the system may correlate the ECAP latency values with user feedback (e.g., patient input indicating loss or reduction in efficacy), changes in stimulation settings, and/or changes in ECAP amplitude to identify how ECAP latency may relate to those other aspects of therapy. These lead shifts over time may occur in paddle leads, cylindrical leads, or any other devices carrying one or more electrodes for delivering stimulation pulses or sensing stimulation pulses.

The following examples are described herein.

Example 1. A system comprising: processing circuitry configured to: control delivery of a first burst of pulses, each pulse of the first burst of pulses defined by a first parameter set; subsequent to delivery of the first burst of pulses, control delivery of a stimulation pulse defined by a second parameter set different from the first parameter set; receive information representative of an evoked compound action potential (ECAP) signal elicited by the stimulation pulse; determine an ECAP characteristic of the ECAP signal, the ECAP characteristic indicative of a latency of one of more nerve fibers activated by the stimulation pulse; determine, based on the ECAP characteristic, one or more parameter values of the first parameter set to generate a third parameter set for a second burst of pulses; and control delivery of the second burst of pulses according to the third parameter set.

Example 2. The system of example 1, wherein: the stimulation pulse is a second stimulation pulse; the ECAP characteristic is a second ECAP characteristic; the latency is a second latency; and the processing circuitry is further configured to receive a first ECAP characteristic indicative of a first latency of the one or more nerve fibers activated by delivery of the first stimulation pulse prior to the second stimulation pulse, wherein the processing circuitry is configured to determine the one or more parameters of the first parameter set by at least determining, based on a comparison of the first latency to the second latency, the one or more parameters of the first parameter set.

Example 3. The system of example 2, wherein the processing circuitry is configured to determine the comparison of the first latency to the second latency.

Example 4. The system of example 3, wherein the processing circuitry is configured to: determine that the second latency is less than the first latency; and responsive to determining that the second latency is less than the first latency, adjust the one or more parameters of the first parameter set to increase a subsequent latency for a subsequent ECAP signal elicited by a subsequent stimulation pulse.

Example 5. The system of any of examples 1 through 4, wherein the first burst of pulses are configured to decrease an activation of a first set of nerve fibers of target tissue instead of a second set of nerve fibers of target tissues, and wherein the first parameter set defines the first burst of pulses comprising a sub-perception threshold intensity.

Example 6. The system of any of examples 1 through 5, wherein the first burst of pulses and the second burst of pulses have a pulse frequency from 200 Hz to 1200 Hz.

Example 7. The system of any of examples 1 through 6, wherein the first parameter set defines the first burst of pulses having an intensity less than a perception threshold.

Example 8. The system of any of examples 1 through 7, wherein the stimulation pulse is one pulse of a plurality of pulses having a pulse frequency from 10 Hz to 60 Hz.

Example 9. The system of any of examples 1 through 8, wherein the one or more parameter values comprises a current amplitude value.

Example 10. The system of any of examples 1 through 9, wherein the one or more parameter values comprises a number of pulses in the second burst of pulses.

Example 11. The system of any of examples 1 through 10, wherein the latency is a first latency, and wherein the processing circuitry is configured to: compare the first latency to a threshold latency; determine that the first latency is greater than the threshold latency; responsive to determining that the first latency is greater than the threshold latency, withhold further bursts of pulses during a first period time in which additional stimulation pulses are delivered; receive information representative of ECAP signals elicited by at least some of the additional stimulation pulses; determine ECAP characteristics of the ECAP signals that are indicative of respective latencies of the one or more nerve fibers activated by the at least some additional stimulation pulses; and responsive to determining that one of the respective latencies becomes shorter than the threshold latency, control delivery of the further bursts of pulses during a second period of time subsequent to the first period of time.

Example 12. The system of any of examples 1 through 11, wherein the processing circuitry is configured to: receive information representative of a plurality of ECAP signals elicited by different stimulation pulses defined by different parameter values; determine, based on the plurality of ECAP signals, a differential ECAP characteristic indicative of the latency of a plurality of different fiber types activated by the different stimulation pulses, the plurality of different fiber types comprising the one or more nerve fibers; determine, based on the differential ECAP characteristic, at least one parameter value of the second parameter set defining the stimulation pulse; and determine, based on the differential ECAP characteristic, at least one parameter value of the first parameter set defining the first burst of pulses.

Example 13. The system of any of examples 1 through 12, wherein the at least one parameter value of the second parameter set comprises a second amplitude value, wherein the at least one parameter value of the first parameter set comprises a first amplitude, and wherein the first amplitude is lower than the second amplitude.

Example 14. The system of any of examples 1 through 13, further comprising an implantable medical device comprising the processing circuitry and stimulation circuitry configured to generate the first burst of pulses, the second burst of pulses, and the stimulation pulse.

Example 15. A method comprising: controlling, by processing circuitry, delivery of a first burst of pulses, each pulse of the first burst of pulses defined by a first parameter set; subsequent to delivery of the first burst of pulses, controlling, by the processing circuitry, delivery of a stimulation pulse defined by a second parameter set different from the first parameter set; receiving, by the processing circuitry, information representative of an evoked compound action potential (ECAP) signal elicited by the stimulation pulse; determining, by the processing circuitry, an ECAP characteristic of the ECAP signal, the ECAP characteristic indicative of a latency of one of more nerve fibers activated by the stimulation pulse; determining, by the processing circuitry and based on the ECAP characteristic, one or more parameter values of the first parameter set to generate a third parameter set for a second burst of pulses; and controlling, by the processing circuitry, delivery of the second burst of pulses according to the third parameter set.

Example 16. The method of example 15, wherein: the stimulation pulse is a second stimulation pulse; the ECAP characteristic is a second ECAP characteristic; the latency is a second latency; the method further comprises receiving a first ECAP characteristic indicative of a first latency of the one or more nerve fibers activated by delivery of the first stimulation pulse prior to the second stimulation pulse, and determining the one or more parameters of the first parameter set comprises determining, based on a comparison of the first latency to the second latency, the one or more parameters of the first parameter set.

Example 17. The method of example 16, further comprising determining the comparison of the first latency to the second latency.

Example 18. The method of example 17, further comprising: determining that the second latency is less than the first latency; and responsive to determining that the second latency is less than the first latency, adjusting the one or more parameters of the first parameter set to increase a subsequent latency for a subsequent ECAP signal elicited by a subsequent stimulation pulse.

Example 19. The method of any of examples 15 through 18, wherein the first burst of pulses are configured to decrease an activation of a first set of nerve fibers of target tissue instead of a second set of nerve fibers of target tissues, and wherein the first parameter set defines the first burst of pulses comprising a sub-perception threshold intensity.

Example 20. The method of any of examples 15 through 19, wherein the first burst of pulses and the second burst of pulses have a pulse frequency from 200 Hz to 1200 Hz.

Example 21. The method of any of examples 15 through 20, wherein the first parameter set defines the first burst of pulses having an intensity less than a perception threshold.

Example 22. The method of any of examples 15 through 21, wherein the stimulation pulse is one pulse of a plurality of pulses having a pulse frequency from 10 Hz to 60 Hz.

Example 23. The method of any of examples 15 through 22, wherein the one or more parameter values comprises a current amplitude value.

Example 24. The method of any of examples 15 through 23, wherein the one or more parameter values comprises a number of pulses in the second burst of pulses.

Example 25. The method of any of examples 15 through 24, wherein the latency is a first latency, and wherein the method further comprises: comparing the first latency to a threshold latency; determining that the first latency is greater than the threshold latency; responsive to determining that the first latency is greater than the threshold latency, withholding further bursts of pulses during a first period time in which additional stimulation pulses are delivered; receiving information representative of ECAP signals elicited by at least some of the additional stimulation pulses; determining ECAP characteristics of the ECAP signals that are indicative of respective latencies of the one or more nerve fibers activated by the at least some additional stimulation pulses; and responsive to determining that one of the respective latencies becomes shorter than the threshold latency, controlling delivery of the further bursts of pulses during a second period of time subsequent to the first period of time.

Example 26. The method of any of examples 15 through 25, further comprising: receiving information representative of a plurality of ECAP signals elicited by different stimulation pulses defined by different parameter values; determining, based on the plurality of ECAP signals, a differential ECAP characteristic indicative of the latency of a plurality of different fiber types activated by the different stimulation pulses, the plurality of different fiber types comprising the one or more nerve fibers; determining, based on the differential ECAP characteristic, at least one parameter value of the second parameter set defining the stimulation pulse; and determining, based on the differential ECAP characteristic, at least one parameter value of the first parameter set defining the first burst of pulses.

Example 27. The method of any of examples 15 through 26, wherein the at least one parameter value of the second parameter set comprises a second amplitude value, wherein the at least one parameter value of the first parameter set comprises a first amplitude, and wherein the first amplitude is lower than the second amplitude.

Example 28. A computer-readable storage medium comprising instructions that, when executed by processing circuitry, cause the processing circuitry to: control delivery of a first burst of pulses, each pulse of the first burst of pulses defined by a first parameter set; subsequent to delivery of the first burst of pulses, control delivery of a stimulation pulse defined by a second parameter set different from the first parameter set; receive information representative of an evoked compound action potential (ECAP) signal elicited by the stimulation pulse; determine an ECAP characteristic of the ECAP signal, the ECAP characteristic indicative of a latency of one of more nerve fibers activated by the stimulation pulse; determine, based on the ECAP characteristic, one or more parameter values of the first parameter set to generate a third parameter set for a second burst of pulses; and control delivery of the second burst of pulses according to the third parameter set.

Example 29. A method comprising: controlling delivery of a stimulation pulse via one or more electrodes; receiving information representative of an evoked compound action potential (ECAP) signal elicited by the stimulation pulse; determining an ECAP characteristic of the ECAP signal, the ECAP characteristic indicative of a latency of one of more nerve fibers activated by the stimulation pulse; and determining, based on the ECAP characteristic indicative of the latency, an implant location for the one or more electrodes.

Example 30. The method of example 29, wherein the latency is a second latency, and wherein the method comprises: determining that the second latency is shorter than a first latency determined from a precious ECAP signal; and determining, based on the second latency being shorter than the first latency, that the one or more electrodes having moved laterally from a midline of a spinal cord.

Example 31. A system configured to perform the method of any of examples 29 and 30.

The techniques described in this disclosure may be implemented, at least in part, in hardware, software, firmware or any combination thereof. For example, various aspects of the described techniques may be implemented within one or more processors or processing circuitry, including one or more microprocessors, digital signal processors (DSPs), application specific integrated circuits (ASICs), field programmable gate arrays (FPGAs), or any other equivalent integrated or discrete logic circuitry, as well as any combinations of such components. The term “processor” or “processing circuitry” may generally refer to any of the foregoing logic circuitry, alone or in combination with other logic circuitry, or any other equivalent circuitry. A control unit including hardware may also perform one or more of the techniques of this disclosure.

Such hardware, software, and firmware may be implemented within the same device or within separate devices to support the various operations and functions described in this disclosure. In addition, any of the described units, circuits or components may be implemented together or separately as discrete but interoperable logic devices. Depiction of different features as circuits or units is intended to highlight different functional aspects and does not necessarily imply that such circuits or units must be realized by separate hardware or software components. Rather, functionality associated with one or more circuits or units may be performed by separate hardware or software components or integrated within common or separate hardware or software components.

The techniques described in this disclosure may also be embodied or encoded in a computer-readable medium, such as a computer-readable storage medium, containing instructions that may be described as non-transitory media. Instructions embedded or encoded in a computer-readable storage medium may cause a programmable processor, or other processor, to perform the method, e.g., when the instructions are executed. Computer readable storage media may include random access memory (RAM), read only memory (ROM), programmable read only memory (PROM), erasable programmable read only memory (EPROM), electronically erasable programmable read only memory (EEPROM), flash memory, a hard disk, a CD-ROM, a floppy disk, a cassette, magnetic media, optical media, or other computer readable media.

1. A system comprising:
processing circuitry configured to:
control delivery of a first burst of pulses, each pulse of the first burst of pulses defined by a first parameter set;
subsequent to delivery of the first burst of pulses, control delivery of a stimulation pulse defined by a second parameter set different from the first parameter set;
receive information representative of an evoked compound action potential (ECAP) signal elicited by the stimulation pulse;
determine an ECAP characteristic of the ECAP signal, the ECAP characteristic indicative of a latency of one of more nerve fibers activated by the stimulation pulse;
determine, based on the ECAP characteristic, one or more parameter values of the first parameter set to generate a third parameter set for a second burst of pulses; and
control delivery of the second burst of pulses according to the third parameter set.

2. The system of claim 1, wherein:
the stimulation pulse is a second stimulation pulse;
the ECAP characteristic is a second ECAP characteristic;
the latency is a second latency; and
the processing circuitry is further configured to receive a first ECAP characteristic indicative of a first latency of the one or more nerve fibers activated by delivery of the first stimulation pulse prior to the second stimulation pulse, wherein the processing circuitry is configured to determine the one or more parameters of the first parameter set by at least determining, based on a comparison of the first latency to the second latency, the one or more parameters of the first parameter set.

3. The system of claim 2, wherein the processing circuitry is configured to determine the comparison of the first latency to the second latency.

4. The system of claim 3, wherein the processing circuitry is configured to:
determine that the second latency is less than the first latency; and
responsive to determining that the second latency is less than the first latency, adjust the one or more parameters of the first parameter set to increase a subsequent latency for a subsequent ECAP signal elicited by a subsequent stimulation pulse.

5. The system of claim 1, wherein the first burst of pulses are configured to decrease an activation of a first set of nerve fibers of target tissue instead of a second set of nerve fibers of target tissues, and wherein the first parameter set defines the first burst of pulses comprising a sub-perception threshold intensity.

6. The system of claim 1, wherein the first burst of pulses and the second burst of pulses have a pulse frequency from 200 Hz to 1200 Hz.

7. The system of claim 1, wherein the first parameter set defines the first burst of pulses having an intensity less than a perception threshold.

8. The system of claim 1, wherein the stimulation pulse is one pulse of a plurality of pulses having a pulse frequency from 10 Hz to 60 Hz.

9. The system of claim 1, wherein the one or more parameter values comprises a current amplitude value.

10. The system of claim 1, wherein the one or more parameter values comprises a number of pulses in the second burst of pulses.

11. The system of claim 1, wherein the latency is a first latency, and wherein the processing circuitry is configured to:
compare the first latency to a threshold latency;
determine that the first latency is greater than the threshold latency;
responsive to determining that the first latency is greater than the threshold latency, withhold further bursts of pulses during a first period time in which additional stimulation pulses are delivered;
receive information representative of ECAP signals elicited by at least some of the additional stimulation pulses;
determine ECAP characteristics of the ECAP signals that are indicative of respective latencies of the one or more nerve fibers activated by the at least some additional stimulation pulses; and
responsive to determining that one of the respective latencies becomes shorter than the threshold latency, control delivery of the further bursts of pulses during a second period of time subsequent to the first period of time.

12. The system of claim 1, wherein the processing circuitry is configured to:
receive information representative of a plurality of ECAP signals elicited by different stimulation pulses defined by different parameter values;
determine, based on the plurality of ECAP signals, a differential ECAP characteristic indicative of the latency of a plurality of different fiber types activated by the different stimulation pulses, the plurality of different fiber types comprising the one or more nerve fibers;
determine, based on the differential ECAP characteristic, at least one parameter value of the second parameter set defining the stimulation pulse; and
determine, based on the differential ECAP characteristic, at least one parameter value of the first parameter set defining the first burst of pulses.

13. The system of claim 1, wherein the at least one parameter value of the second parameter set comprises a second amplitude value, wherein the at least one parameter value of the first parameter set comprises a first amplitude, and wherein the first amplitude is lower than the second amplitude.

14. The system of claim 1, further comprising an implantable medical device comprising the processing circuitry and stimulation circuitry configured to generate the first burst of pulses, the second burst of pulses, and the stimulation pulse.

15. A computer-readable storage medium comprising instructions that, when executed by processing circuitry, cause the processing circuitry to perform the functions of claim 1.

16. A method comprising:
controlling, by processing circuitry, delivery of a first burst of pulses, each pulse of the first burst of pulses defined by a first parameter set;
subsequent to delivery of the first burst of pulses, controlling, by the processing circuitry, delivery of a stimulation pulse defined by a second parameter set different from the first parameter set;
receiving, by the processing circuitry, information representative of an evoked compound action potential (ECAP) signal elicited by the stimulation pulse;
determining, by the processing circuitry, an ECAP characteristic of the ECAP signal, the ECAP characteristic indicative of a latency of one of more nerve fibers activated by the stimulation pulse;
determining, by the processing circuitry and based on the ECAP characteristic, one or more parameter values of the first parameter set to generate a third parameter set for a second burst of pulses; and
controlling, by the processing circuitry, delivery of the second burst of pulses according to the third parameter set.

17. The method of claim 16, wherein:
the stimulation pulse is a second stimulation pulse;
the ECAP characteristic is a second ECAP characteristic;
the latency is a second latency;
the method further comprises receiving a first ECAP characteristic indicative of a first latency of the one or more nerve fibers activated by delivery of the first stimulation pulse prior to the second stimulation pulse, and
determining the one or more parameters of the first parameter set comprises determining, based on a comparison of the first latency to the second latency, the one or more parameters of the first parameter set.

18. The method of claim 17, further comprising:
determining the comparison of the first latency to the second latency;
determining that the second latency is less than the first latency; and
responsive to determining that the second latency is less than the first latency, adjusting the one or more parameters of the first parameter set to increase a subsequent latency for a subsequent ECAP signal elicited by a subsequent stimulation pulse.

19. The method of claim 16, wherein the first burst of pulses are configured to decrease an activation of a first set of nerve fibers of target tissue instead of a second set of nerve fibers of target tissues, and wherein the first parameter set defines the first burst of pulses comprising a sub-perception threshold intensity.

20. The method of claim 16, wherein the first burst of pulses and the second burst of pulses have a pulse frequency from 200 Hz to 1200 Hz.

21. The method of claim 16, wherein the first parameter set defines the first burst of pulses having an intensity less than a perception threshold.

22. The method of claim 16, wherein the stimulation pulse is one pulse of a plurality of pulses having a pulse frequency from 10 Hz to 60 Hz.

23. The method of claim 16, wherein the latency is a first latency, and wherein the method further comprises:
comparing the first latency to a threshold latency;
determining that the first latency is greater than the threshold latency;
responsive to determining that the first latency is greater than the threshold latency, withholding further bursts of pulses during a first period time in which additional stimulation pulses are delivered;
receiving information representative of ECAP signals elicited by at least some of the additional stimulation pulses;
determining ECAP characteristics of the ECAP signals that are indicative of respective latencies of the one or more nerve fibers activated by the at least some additional stimulation pulses; and
responsive to determining that one of the respective latencies becomes shorter than the threshold latency, controlling delivery of the further bursts of pulses during a second period of time subsequent to the first period of time.

24. The method of claim 16, further comprising:
receiving information representative of a plurality of ECAP signals elicited by different stimulation pulses defined by different parameter values;
determining, based on the plurality of ECAP signals, a differential ECAP characteristic indicative of the latency of a plurality of different fiber types activated by the different stimulation pulses, the plurality of different fiber types comprising the one or more nerve fibers;
determining, based on the differential ECAP characteristic, at least one parameter value of the second parameter set defining the stimulation pulse; and
determining, based on the differential ECAP characteristic, at least one parameter value of the first parameter set defining the first burst of pulses.

25. A computer-readable storage medium comprising instructions that, when executed by processing circuitry, cause the processing circuitry to:
control delivery of a first burst of pulses, each pulse of the first burst of pulses defined by a first parameter set;
subsequent to delivery of the first burst of pulses, control delivery of a stimulation pulse defined by a second parameter set different from the first parameter set;
receive information representative of an evoked compound action potential (ECAP) signal elicited by the stimulation pulse;
determine an ECAP characteristic of the ECAP signal, the ECAP characteristic indicative of a latency of one of more nerve fibers activated by the stimulation pulse;
determine, based on the ECAP characteristic, one or more parameter values of the first parameter set to generate a third parameter set for a second burst of pulses; and
control delivery of the second burst of pulses according to the third parameter set.
